(19) United States
(12) Patent Application Publication
(10) Pub. No.: US 2026/0102606 A1
(43) Pub. Date: Apr. 16, 2026

(54) MEDICAL DEVICE INCLUDING FLEXIBLE ELECTRODE ARRAY

(71) Applicant: Covidien LP, Mansfield, MA (US)

(72) Inventors: Robert L. Olson, White Bear Lake, MN (US); Dale F. Seeley, Spring Park, MN (US)

(21) Appl. No.: 19/358,531

(22) Filed: Oct. 15, 2025

Related U.S. Application Data
(60) Provisional application No. 63/707,494, filed on Oct. 15, 2024.

(51) Int. Cl. A61N 1/05; A61N 1/36

(52) U.S. Cl. CPC A61N 1/0504; A61N 1/36053

(57) ABSTRACT

An endovascular device includes an elongated body, an expandable structure, and an electrode array. The elongated body is configured to be introduced into a blood vessel of a patient. The electrode array includes a flexible polymer packaging and one or more electrodes electrically coupled to the elongated body. The flexible polymer packaging is coupled to expandable structure and the one or more electrodes. In some examples, the expandable structure is a non-tubular expandable structure configured to bend around a longitudinal axis. In some examples, the expandable structure is a tubular expandable structure, and the one or more electrodes are embedded in the flexible polymer packaging. In some examples, the expandable structure is mechanically coupled to the elongated body and configured to be positioned within a stent.

### CROSS-REFERENCE TO RELATED APPLICATIONS

This application claims the benefit of and priority to U.S. Provisional Patent Application Serial No. 63/707,494 filed October 15, 2024, the entire disclosure of which is incorporated by reference herein.

### TECHNICAL FIELD

This disclosure relates to medical devices, such as endovascular devices, for electrical stimulation therapy.

### BACKGROUND

Medical devices, such as electrical stimulation devices, may be used in different therapeutic applications, such as vagus nerve stimulation (VNS) and/or deep brain stimulation (DBS). A medical device may be used to deliver therapy to a patient to treat a variety of symptoms or patient conditions. In some therapy systems, an external or an implantable electrical stimulator delivers electrical stimulation therapy to a target tissue site within a patient with the aid of one or more electrodes and/or senses one or more patient parameters with the aid of the one or more electrodes.

### SUMMARY

This disclosure describes medical devices, such as endovascular devices, that include electrode arrays having flexible polymers for attachment to expandable structures. An endovascular device, such as a lead, includes an elongated body configured to be introduced into a blood vessel of a patient. An expandable structure, such as a stent, and an electrode array are positioned at a distal end of the elongated body and configured to be positioned within the blood vessel for therapy or sensing. The electrode array includes one or more electrodes and a flexible polymer packaging that is coupled to both the expandable structure and the electrodes. The flexible polymer packaging firmly couples the electrodes to the expandable structure and accommodates expansion and contraction of the expandable structure, such that electrodes may be accurately positioned once the expandable structure is in place.

In some examples, the flexible polymer packaging includes a support layer that couples the electrode array to the expandable structure and an encapsulation layer that encapsulates electrode wires, and optionally adheres the electrodes to the flexible polymer packaging. During fabrication of the electrode array, the support layer may be molded into or onto the expandable structure, such that the support layer is securely attached to the expandable structure. The one or more electrodes and electrode wires may be positioned on the support layer, such as at a location corresponding to a treatment site once the medical device is positioned within a vessel of a patient. The elongated body may be electrically coupled to the electrode array and, optionally, mechanically coupled to the expandable structure. After attachment of the elongated body, the encapsulation layer may be subsequently applied to the support layer to encapsulate the electrode wires and/or embed the electrodes. The resulting endovascular device may be positioned within the vessel of the patient, communicatively coupled to an implantable therapy device, and controlled to deliver therapy to the patient or to sense one or more patient parameters (e.g., brain signals) via the one or more electrodes. Upon deployment of the expandable member, the electrodes may expand or contract with expansion or contraction of the expandable structure, and may remain secured to the expandable structure during delivery of the therapy.

In some examples, the expandable structure is a non-tubular expandable structure configured to bend around a longitudinal axis. For example, the expandable structure may be a planar stent preform that can be subsequently formed into a cylindrical shape and delivered to the treatment site. The planar stent preform may be a flexible metal template, and the flexible polymer packaging may include one or more polymer sheets molded onto the flexible metal template. The planar stent preform may be configured to self-deploy into a tubular shape once positioned at the treatment site. For example, the planar stent preform may be configured to collapse into a small form factor and, once unconstrained, expand to the vessel.

In some examples, the expandable structure is a tubular expandable structure, such as a tubular stent, and both expandable structure and the electrodes are embedded in the flexible polymer packaging. For example, one or more arrays of electrodes may be discretely positioned on the stent and electrically coupled to the elongated body through one or more insulated wires. In some examples, the electrode array include more than one array, such that each array is electrically coupled to the elongated body through a separate insulated wire to accommodate expansion or contraction of the tubular expandable structure.

In some examples, the expandable structure is mechanically coupled to the elongated body and configured to be positioned within an existing stent in the patient. For example, the expandable structure may be coupled to the electrode array and configured to position the electrode array into or around the existing stent. The flexible polymer packaging may be configured to expand or contract to fit into the existing stent and contact the vessel supported by the stent.

In some examples, electrode array is formed prior to positioning on the expandable member. For example, the flexible polymer packaging may be formed as a separate structure, such as a tubular sleeve, and subsequently positioned around the expandable member.

In these various ways, medical devices described herein may have greater integrity and more easily and/or accurately manufactured compared to other medical devices that do not include a flexible polymer packaging coupled to electrodes and the expandable structure.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1A is a conceptual diagram illustrating an example therapy system including an endovascular device configured to deliver electrical stimulation therapy to a target tissue site of a patient and/or sense a patient parameter from an endovascular location.

FIG. 1B is a functional block diagram illustrating components of an example medical device of the therapy system of FIG. 1A.

FIG. 2A is a perspective view of a distal portion of an example medical device in a pre-delivery configuration.

FIG. 2B is a cross-sectional axis view diagram of the example medical device of FIG. 2A in the pre-delivery configuration.

FIG. 2C is a cross-sectional axis view diagram of the example medical device of FIG. 2A in a delivery configuration.

FIG. 2D is an expanded cross-sectional top view diagram of the example medical device of FIG. 2A.

FIG. 3 is a flowchart of an example method for fabricating a medical device.

FIG. 4A is a perspective view of a flexible metal template used for forming an example medical device.

FIG. 4B is a perspective view of a support layer overlying the flexible metal template of FIG. 4A.

FIG. 4C is a perspective view diagram of a plurality of electrodes overlying the support layer of FIG. 4B.

FIG. 4D is a perspective view diagram of an encapsulation layer overlying the support layer of FIG. 4C.

FIG. 5A is a perspective view of a distal portion of an example endovascular device.

FIG. 5B is a top view of the example endovascular device of FIG. 5A.

FIG. 5C is a cross-sectional axis view diagram of the example endovascular device of FIG. 5B.

FIG. 6 is a flowchart of an example method for fabricating an endovascular device.

FIG. 7A is a top view of a support layer overlying a tubular expandable structure.

FIG. 7B is a top view of a plurality of electrodes overlying the support layer of FIG. 7A.

FIG. 7C is a top view of an encapsulation layer overlying the support layer of FIG. 7B.

FIG. 8A is a perspective view of a distal portion of an example endovascular device.

FIG. 8B is a side view of the endovascular device of FIG. 8A positioned within a stent.

FIG. 8C is a cross-sectional axis view diagram of the example endovascular device of FIG. 8B.

FIG. 8D is a side view of an endovascular device that includes an alternative electrode array.

FIG. 9 is a flowchart of an example method for fabricating an endovascular device.

FIG. 10A is a perspective view of a distal portion of an example endovascular device.

FIG. 10B is a top view of the example endovascular device of FIG. 10A.

FIG. 10C is a cross-sectional axis view diagram of the example endovascular device of FIG. 10B.

FIG. 11 is a flowchart of an example method for fabricating an endovascular device.

FIG. 12A is a top view of a plurality of electrodes and electrode traces overlying a support layer.

FIG. 12B is a top view of an encapsulation layer overlying the support layer of FIG. 12A.

FIG. 12C is a top view of a flexible polymer packaging coupled to an expandable member.

### DETAILED DESCRIPTION

This disclosure describes devices, systems, and methods relating to delivery of electrical stimulation therapy, such as vagus nerve stimulation (VNS), deep brain stimulation (DBS), and/or sensing one or more patient parameters (e.g., nerve activity from one more nerves, cardiac signals, muscle activation signals, brain signals and/or other physiological parameters, such as impedance, electroencephalogram (EEG), evoked potentials, local field potentials, etc.) from an endovascular location. Example endovascular locations that can be used for electrical stimulation therapy (e.g., VNS therapy) and/or sensing using the devices described herein include an internal jugular vein (IJV). Example endovascular locations that can be used to access the brain sites for electrical stimulation therapy (e.g., DBS) and/or sensing using the devices described herein include any suitable cranial blood vessel (also referred to herein as a cerebral blood vessel or neurovasculature, which can include a vein or an cranial artery), such as, but not limited to, the thalamostriate vein, the internal cerebral vein, the basal vein of Rosenthal, the inferior sagittal sinus, the superior sagittal sinus, or the anterior choroidal artery. While this disclosure is primarily directed to examples of VNS and/or sensing via applicable endovascular locations (e.g., the internal jugular vein), it should be understood that the devices, systems, and techniques may be adapted for DBS, other kinds of brain stimulation, peripheral nerve stimulation, or electrical stimulation and/or sensing of any nerve tissue that can be done via an endovascular location.

VNS has been proposed for use to manage one or more patient conditions, such as to control an inflammatory response in patients. Stimulating the vagus nerve may dampen the inflammatory response and associated cytokine response. In some examples, inflammatory cytokines are modulated up or down via stimulation. In addition, VNS may assist in stroke rehabilitation and limit ischemia reperfusion injury. After a myocardial infarct or stroke, reperfusion therapies (surgery or drugs) are given to restore blood flow. However, due to the restoration of blood, flow induced local damage occurs, including ischemia reperfusion injury. This injury may induce local accumulations of chemical mediators such as reactive oxygen species (ROS) production, inflammatory cytokines, bradykinin, etc., which can further affect inflammation. Such inflammatory compounds may trigger sensory signaling, which can lead to a reduced organ vagus activity and sympathetic overdrive. Vagus nerve stimulation may treat reperfusion damage as the inflammatory state may be lowered by increasing parasympathetic drive.

DBS has been proposed for use to manage one or more patient conditions. For example, DBS can be used to alleviate, and in some cases, eliminate symptoms associated with movement disorders, other neurodegenerative impairment, seizure disorders, psychiatric disorders (e.g., mood disorders), or the like. Movement disorders may be found in patients with Parkinson’s disease, multiple sclerosis, and cerebral palsy, among other conditions, and can be associated with disease or trauma. DBS can be delivered to one or more target sites in a brain of a patient to help a patient with muscle control and minimize movement problems, such as rigidity, bradykinesia (i.e., slow physical movement), rhythmic hyperkinesia (e.g., tremor), nonrhythmic hyperkinesia (e.g., tics) or akinesia (i.e., a loss of physical movement).

In the case of seizure disorders, DBS can be delivered to one or more target sites in a brain of a patient to reduce the frequency or severity of seizures, or even help prevent the occurrence of seizures. In the case of psychiatric disorders, DBS can be delivered to help minimize or even eliminate symptoms associated with major depressive disorder (MDD), bipolar disorder, anxiety disorders, post-traumatic stress disorder, dysthymic disorder, or obsessive-compulsive disorder (OCD). DBS can also reduce the symptoms of Parkinson’s disease, dystonia, or cerebellar outflow tremor.

In the examples described herein, an endovascular device includes one or more electrodes that are carried by an expandable structure at a distal portion of an elongated body (e.g., a medical lead). The expandable structure is configured to transform between a delivery (e.g., compressed) configuration and a deployed (e.g., expanded) configuration. The endovascular device may be electrically coupled to a therapy device configured to generate electrical stimulation and/or sense a patient parameter via the electrodes of the endovascular device. The electrodes may be carried by or otherwise disposed on an expandable structure, which may be configured to orient the electrodes and/or anchor the electrodes at a particular location in the vasculature of the patient.

Rather than directly attached the electrodes to the expandable structure, endovascular devices described herein include a flexible polymer packaging that indirectly couples the electrodes to the expandable structure. The flexible polymer packaging is configured to accommodate movement of the expandable structure, such as expansion or contraction, such that electrodes may remain positioned at a desired location on the expandable structure. The flexible polymer packaging may also insulate electrode wires and/or embed electrodes. In some examples, the flexible polymer packaging may include two layers that may be formed on the expandable structure and package components of the electrode array in situ. The flexible polymer packaging may be adaptable to a variety of expandable structures, including non-tubular expandable structures, tubular expandable structures, and helical expandable structures.

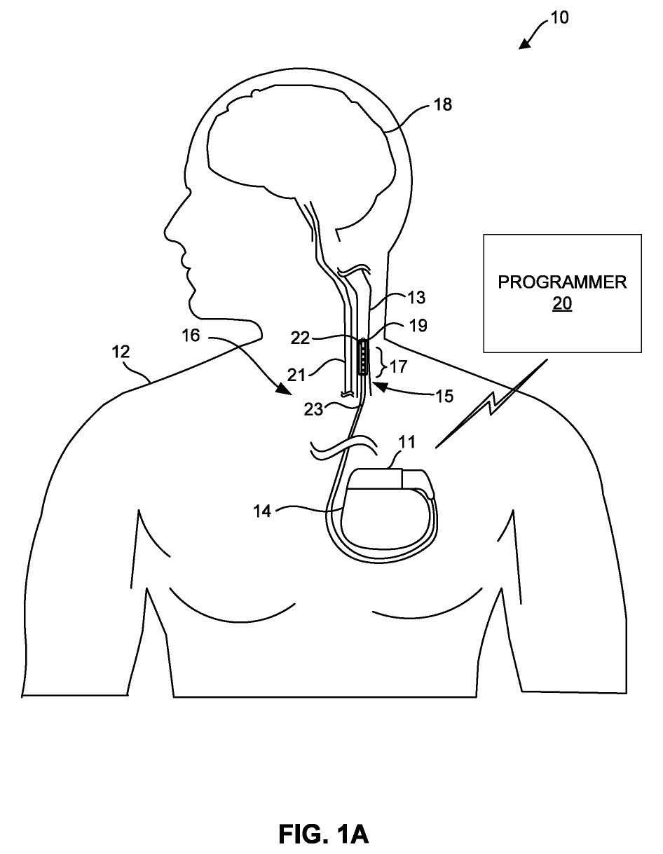

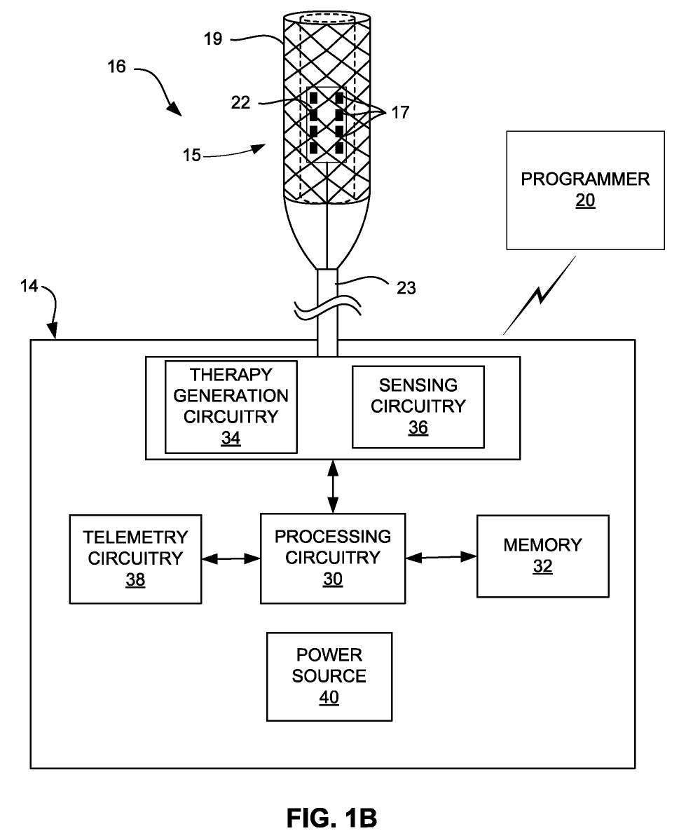

Endovascular devices described herein, having enhanced integrity and accurate electrode positioning, may be particularly useful for electrical stimulation therapy, in which the endovascular device is positioned within a patient for a relatively long period of time. FIG. 1A is a conceptual diagram illustrating an example therapy system 10 configured to deliver electrical stimulation therapy to a target tissue site of a patient 12 or sense a patient parameter from an endovascular location. Patient 12 ordinarily will be a human patient; however, in some cases, therapy system 10 is applied to other mammalian or non-mammalian non-human patients. Therapy system 10 includes a therapy device 14 and an endovascular device 16. In the example shown in FIG. 1A, therapy device 14 is configured to deliver electrical stimulation therapy (e.g., VNS) to a vagus nerve 21 of patient 12 and/or sense bioelectric signals via electrodes 17. However, in other examples, therapy system 10 and/or therapy device 14 is configured to deliver electrical stimulation therapy (e.g., DBS) to brain 18 of patient 12 and/or sense bioelectrical brain signals in brain 18 via electrodes 17.

Endovascular device 16 is positioned in a jugular vein 13 of patient 12 such that one or more electrodes 17 are located proximate to a target tissue site. In particular, electrodes 17 are positioned to deliver electrical stimulation therapy to and/or sense signals from nerves surrounding jugular vein 13, including (but not limited to) vagus nerve 21. Endovascular device 16 includes an expandable structure 19 at a distal portion 15 of endovascular device 16 which may help hold electrodes 17 in apposition with a vessel wall (e.g., of jugular vein 13). In some examples, expandable structure 19 is at a distalmost end of endovascular device 16. Endovascular device 16 includes an elongated body 23 that electrically couples therapy device 14 to electrodes 17. Therapy device 14 can provide electrical stimulation to one or more regions surrounding jugular vein 13 in order to manage a condition of patient 12, such as to mitigate the severity or duration of the patient condition.

Endovascular device 16 includes any suitable medical device configured to deliver electrical stimulation signals to tissue proximate electrodes 17. For example, endovascular device 16 can be (or include) a medical lead, a catheter, a guidewire, or another elongated body carrying electrodes 17 and configured to be electrically coupled to therapy device 14 via an electrically conductive pathway that runs between therapy device 14 and electrodes 17. Endovascular device 16 has any suitable length that enables connection to therapy device 14 either directly or indirectly, e.g., a length of 150 centimeters (cm) to 250 cm, such as 200 cm. Further, endovascular device 16 has a suitable length (e.g., as measured along a longitudinal axis of endovascular device 16) for accessing a target tissue site within the patient from a vascular access point. In examples in which endovascular device 16 accesses the jugular vein 13 and/or vasculature in a brain 18 of patient 12 from a femoral artery access point at the groin of the patient, endovascular device 16 has a length of about 100 cm to about 200 cm, although other lengths may be used. As used herein, “about” may indicate the exact value or nearly the exact value to the extent permitted by manufacturing tolerances. “About” can also refer to a certain percentage of the recited value (e.g., within about 1%, 5%, or 10%).

Endovascular device 16 is configured to be introduced in the vasculature of patient 12, such as to access jugular vein 13 and/or relatively more distal locations in a patient, such as the middle cerebral artery (MCA) in a brain of a patient. Elongated body 23 is structurally configured to be relatively flexible, pushable, and relatively kink- and buckle-resistant, so that it may resist buckling when a pushing force is applied to a relatively proximal portion to advance endovascular device 16 distally through vasculature, and so that it may resist kinking when traversing around a tight turn in the vasculature. Kinking and/or buckling of may hinder a clinician’s efforts to push the elongated body distally, e.g., past a turn. In some examples, endovascular device 16 includes one or more radiopaque components (e.g., platinum bands) proximate electrodes 17 and/or expandable structure 19.

Instead of or in addition to the elongated body of endovascular device 16 being configured for intravascular navigation to a cerebral blood vessel to deliver electrical stimulation therapy or sense a patient parameter, endovascular device 16 can be navigated through vasculature (e.g., to jugular vein 13, brain 18, or other target tissue sites) with the aid of a guide member. The guide member can include an outer catheter, an inner catheter, a guide extension catheter, a guidewire, or the like or combination thereof.

In some examples, more than one endovascular device 16 is implanted within patient 12 to provide support, provide stimulation to, and/or sense multiple anatomical regions, including one or more of both the left and right jugular veins, as well as in locations of brain 18. For example, two or more of endovascular device 16, which may be paired with one or more of therapy device 14, may be configured for bilateral stimulation and/or sensing (e.g., of the left jugular vein and a right jugular vein). As another example, an endovascular device, such as a stent, may be positioned in patient 12 prior to positioning endovascular device 16, such that endovascular device 16 may be used in conjunction with a support or other function of the additional endovascular device. Endovascular device 16, including electrodes 17 and/or expandable structure 19, can be implanted in a blood vessel for chronic therapy delivery and/or chronic sensing (e.g., on the order of months or even years) or for more temporary therapy delivery and/or sensing (e.g., on the order of days, such as less than a month or less than 6 months). Temporary therapy delivery may include one or more trial periods, such as to determine, evaluate, or confirm an efficacy of stimulation and/or sensing.

The electrical stimulation therapy described herein (e.g., VNS) may be used to treat various patient conditions, such as, a variety of illnesses including, but not limited to: reperfusion damage, cardiac ischemia, brain ischemia, stroke, traumatic brain injury, surgical or non-surgical acute kidney injury, inability of the intestine (bowel) to contract normally and move waste out of the body, postoperative ileus, postoperative cognitive decline or postoperative delirium, asthma, sepsis, bleeding control, myocardial infarction reduction, dysmotility, and obesity. Treating any of these diseases may improve patient outcomes by shortening length of hospital stays and reducing medical costs.

The vasculature into which endovascular device 16 may be inserted and/or guided includes, but is not limited to, veins or arteries. For example, endovascular device 16 can be navigated from a vasculature access site (e.g., in the femoral artery, the radial artery, or another suitable access site) to one or more of a jugular vein (e.g., internal jugular vein and/or external jugular vein), a carotid artery (e.g., internal carotid artery, external carotid artery, and/or common carotid artery), as well as brain targets including the thalamostriate vein, the internal cerebral vein, the basal vein of Rosenthal, the inferior/superior sagittal sinus, the anterior choroidal artery, or any related combinations thereof.

A clinician can also select a particular blood vessel to position electrodes 17 within, such as to avoid certain regions to minimize or even eliminate adverse effects. For example, electrodes 17 can be oriented or positioned relative to vagus nerve 21 to avoid inadvertently providing electrical stimulation to anatomical regions (e.g., undesired anatomical regions) near the targeted anatomical region.

Endovascular device 16 is configured to be delivered to one or more target sites in vasculature of patient 12. Rather than introducing endovascular device 16 into tissue in close proximity with vagus nerve 21 through an incision in the neck or chest area of patient 12, endovascular device 16 is configured to be navigated proximate to a target electrical stimulation site via vasculature of patient 12. The endovascular delivery of endovascular device 16 to target sites can help minimize the invasiveness of therapy system 10.

Electrodes 17 are positioned on (e.g., coupled to, defined by, or otherwise carried by) expandable structure 19 of endovascular device 16 via a flexible polymer packaging 22. Expandable structure 19 is configured to expand radially outwards from a relatively low-profile (e.g., radially compressed) delivery configuration to a deployed configuration. This may enable electrodes 17 to be held in apposition with a blood vessel wall and promote tissue ingrowth around electrodes 17 along the vessel wall (while still leaving a patent lumen to enable blood flow through the blood vessel, through expandable structure 19, despite implantation of endovascular device 16), which can reduce the overall power needed to deliver efficacious electrical stimulation therapy to a target tissue site, and help secure electrodes 17 in place in the blood vessel for chronic therapy delivery.

Therapy device 14 can be an external medical device or an implantable medical device that includes electrical stimulation circuitry configured to generate and deliver electrical stimulation therapy to patient 12 and/or sensing circuitry configured to sense a patient parameter (e.g., a physiological signal) via one or more electrodes 17 of endovascular device 16. In the example shown in FIG. 1A, endovascular device 16 is directly or indirectly mechanically and electrically coupled to therapy device 14 via a header (or connector block or connector) 11 of therapy device 14, which defines a plurality of electrical contacts in one or more feedthrough portions for electrically coupling electrodes 17 to electrical stimulation generation circuitry and/or sensing circuitry within therapy device 14. In some examples, header 11 includes multiple feedthrough portions, which may be respectively configured for receiving one of multiple portions of endovascular device 16. Endovascular device 16 may be coupled to header 11 with the aid of a lead extension, or may be directly mechanically and/or electrically connected to therapy device 14 via header 11.

In some examples, therapy device 14 is configured to be implanted in patient 12 in any suitable location, such as a location in a pectoral region. In other examples, therapy device 14 is configured to be external to patient 12. Endovascular device 16 may be, for example, implanted within a vein (e.g., jugular vein 13) and one or more proximal wires/leads can remain within the venous system until they exit the venous system, such as through the subclavian vein in the chest or the internal jugular vein in the neck for implant in the pectoral region. In yet other examples, some or all of therapy device 14 is configured to be implanted in the vasculature, e.g., as part of endovascular device 16.

As shown in FIG. 1A, system 10 may also include a programmer 20, which may be a handheld device, portable computer, or workstation that provides a user interface to a user, for example a clinician or other user, such as a patient. The user may interact with the user interface to program electrical stimulation parameters for therapy device 14.

With the aid of programmer 20 or another computing device, a clinician may select values for therapy parameters for controlling therapy delivery by therapy system 10. The values for the therapy parameters may be organized into a group of parameter values referred to as a “therapy program” or “therapy parameter set.” In the case of electrical stimulation, the therapy parameters may include an electrode combination, a power, and an amplitude, which may be a current or voltage amplitude, and, if therapy device 14 delivers electrical pulses, a pulse width, and a pulse rate for stimulation signals to be delivered to the patient. Other example therapy parameters include a slew rate, duty cycle, and phase of the electrical stimulation signal.

An electrode combination may include a selected subset of one or more electrodes 17 located on one or more implantable endovascular devices 16 coupled to therapy device 14. The electrode combination may also refer to the polarities of the electrodes in the selected subset. By selecting particular electrode combinations, a user may target particular tissue sites (e.g., anatomic structures) within patient 12. In addition, by selecting values for slew rate, duty cycle, phase amplitude, pulse width, and/or pulse rate, the user can attempt to generate an efficacious therapy for patient 12 that is delivered via the selected electrode subset.

Whether programmer 20 is configured for clinician or patient use, programmer 20 may be configured to communicate with therapy device 14 or any other computing device via wireless or a wired communication. Programmer 20, for example, may communicate via wireless communication with therapy device 14 using radio frequency (RF) telemetry techniques. Programmer 20 may also communicate with another programmer or computing device via a wired or wireless connection using any of a variety of local wireless communication techniques, such as RF communication according to the 802.11 or Bluetooth specification sets, infrared communication according to the Infrared Data Association (IRDA) specification set, or other standard or proprietary telemetry protocols. Programmer 20 may also communicate with another programming or computing device via a wired or wireless communication technique.

In some examples, in addition to or instead of delivering electrical stimulation to a target location (e.g., vagus nerve 21), therapy device 14 or another device senses one or more patient parameters, such as bioelectrical signals, either using electrodes 17 or other types of sensors that are carried by endovascular device 16. Bioelectric signals can be sensed, and indications of sensed signals can be used by clinicians to make clinically relevant decision. In other examples, sense bioelectric signals are used as part of continuous feedback system in which therapy device 14 adjusts one or more therapy parameter values based on sensed bioelectrical signals. Example bioelectric signals are described in further detail below with reference to FIG. 1B.

In some examples, therapy device 14 is configured to generate and deliver a suitable electrical stimulation signal, which can be a continuous time signal (e.g., a sinusoidal waveform or the like) or a plurality of pulses. In some examples, the electrical stimulation waveform generated by therapy device 14 and delivered by one or more of electrodes 17 is a charge balanced, biphasic waveform. In some examples, such an electrical stimulation waveform consists of periodic pulses or otherwise include periodic pulses, or can include a continuous time waveform.

As noted above, in some examples, one or more electrodes 17 are positioned on expandable structure 19 via flexible polymer packaging 22. In some examples, one or more sensors that are different from electrodes 17 are positioned on the same expandable structure (e.g., expandable structure 19) as one or more electrodes 17 or on a different expandable structure (e.g., a structure similar to or different from expandable structure 19) of endovascular device 16. Expandable structure 19 can have any suitable configuration that enables endovascular device 16 to assume a relatively low-profile configuration (also referred to herein as a “delivery” or “compressed” configuration in some examples) to facilitate delivery through vasculature to a target tissue site and expand radially outwards (relative to a central longitudinal axis of endovascular device 16) to position the one or more electrodes 17 closer to target tissue.

In some examples, expandable structure 19 is configured to expand radially outwards with sufficient force and to a cross-sectional dimension (e.g., a diameter) sufficient to position the one or more electrodes 17 in apposition with a blood vessel wall. As mentioned above, positioning one or more electrodes 17 in apposition with a blood vessel wall may help promote tissue ingrowth around electrodes 17, which can reduce the impedance and the overall power needed to deliver efficacious electrical stimulation therapy to a target tissue site, and help secure electrodes 17 in place in the blood vessel for chronic (e.g., on the order of months or even years) therapy delivery. Fixing endovascular device 16 in place within the blood vessel via the tissue ingrowth or, in some examples, using another fixation structures/anchoring mechanisms, such as tines, coils barbs, or the like, can also help reduce the possibility of thrombosis.

Expandable structure 19 can be configured to expand radially outwards using any suitable technique and configuration. In some examples, expandable structure 19 includes a shape memory (e.g., nitinol) material that enables the expandable structure to assume a predetermined shape in the absence of a force (e.g., a compressive or tensile force) holding expandable structure 19 in a relatively low-profile delivery configuration. For example, expandable structure 19 can be configured to expand radially outwards upon deployment from an outer sheath (e.g., an outer catheter), or upon the proximal withdrawal of a straightening element (e.g., a guidewire or a mandrel) positioned in an inner lumen of the endovascular device 16. In some examples, expandable structure 19 is configured to expand radially outwards in response to proximal withdrawal of a pull member attached to a distal portion of the endovascular device 16, in response to a distal movement of an elongated control member attached to the expandable structure, or with the aid of a balloon or the like.

Expandable structure 19 can have any suitable configuration in its deployed (e.g., expanded) configuration. In some examples herein, expandable structure 19 is a tubular expandable structure that includes a plurality of interconnected struts to form a structure configured to expand radially outward (e.g., from a central longitudinal axis of expandable structure 19). For example, expandable structure 19 can include a tubular member, a basket, include one or more splines or arms configured to expand radially outwards, define one or more loops, define a helical or spiral element, or the like or combinations thereof, when in the deployed configuration. One or more expandable structures 19 may be disposed at various positions along endovascular device 16 (e.g., at one or more longitudinal positions along endovascular device 16). Expandable structure 19 can be formed from a plurality of structural elements (e.g., braided or coupled together) or can be a unitary structure (e.g., a laser cut nitinol tube).

Endovascular device 16 may have any suitable configuration for delivering electrical stimulation to a target tissue site in patient 12 or sensing a patient parameter from an endovascular location (e.g., jugular vein 13). In some examples, endovascular device includes a first subset of electrodes of electrodes 17 configured for delivering electrical stimulation therapy and a second subset of electrodes of electrodes 17 configured to for sensing one or more patient parameters. In some examples, some or all electrodes of electrodes 17 are configured for both electrical stimulation therapy and for sensing one or more patient parameters. Endovascular device 16 can include any suitable number of electrodes 17 and/or combination of different kinds of electrodes.

FIG. 1B is a functional block diagram illustrating components of an example therapy device 14, which is configured to generate and deliver electrical stimulation therapy to patient 12 and, in some examples, sense one or more patient parameters, such as bioelectrical signals or other physiological parameter of patient 12. Therapy device 14 includes processing circuitry 30, memory 32, therapy generation circuitry 34, sensing circuitry 36, telemetry circuitry 38, and power source 40.

Therapy generation circuitry 34 includes any suitable configuration (e.g., hardware) configured to generate and deliver electrical stimulation signals to target tissue (e.g., vagus nerve 21) in patient 12. Processing circuitry 30 is configured to control therapy generation circuitry 34 to generate and deliver electrical stimulation therapy via electrodes 17 of endovascular device 16. The therapy parameter values may be selected based on the patient condition being addressed, as well as the target tissue site in patient 12 for the electrical stimulation therapy. The electrical stimulation therapy can be provided via stimulation signals of any suitable form, such of stimulation pulses or continuous-time signals (e.g., sine waves).

Sensing circuitry 36 is configured to sense a physiological parameter of a patient. Sensing circuitry 36 may include any sensing hardware configured to sense a physiological parameter of a patient, such as, but not limited to, one or more electrodes, optical receivers, pressure sensors, or the like. The one or more sensing electrodes can be the same or different from electrodes 17 configured to deliver electrical stimulation therapy. In some examples, processing circuitry 30 stores the sensed physiological parameters in memory 32 or transmits the sensed parameters to another device via telemetry circuitry 38. In addition, in some examples, processing circuitry 30 can use the sensed physiological signals to control therapy delivery by therapy generation circuitry 34, e.g., the timing of the therapy delivery or one or more characteristics (e.g., parameters values) of the electrical simulation signal generated by therapy generation circuitry 34.

In some examples, sensing circuitry 36 is configured to sense a bioelectrical signal, which otherwise may be referred to as a patient parameter, via one or more electrodes 17 (e.g., all or a subset of electrodes 17). Thus, electrodes 17 can be configured to receive or transmit energy (e.g., current). In some examples, such as those in which electrodes 17 are placed proximate vagus nerve 21 (FIG. 1), example bioelectric signals include muscle activation signals (e.g., laryngeal muscle activation), electrocardiogram (ECG), intracardiac electrogram (EGM), electromyogram (EMG). In other examples, such as those in which electrodes 17 are placed in or otherwise proximate brain 18, example bioelectrical signals include brain signals such as an EEG signal, an electrocorticogram (ECoG) signal, a signal generated from measured field potentials within one or more regions of brain 18, action potentials from single cells within brain 18 (referred to as “spikes”), or evoked potentials. Determining action potentials of single cells within brain 18 may require resolution of bioelectrical signals to the cellular level and provides fidelity for fine movements, i.e., a bioelectrical signal indicative of fine movements (e.g., slight movement of a finger). In examples in which endovascular device 16 is configured to sense an evoked potential, endovascular device 16 may also be configured to generate a stimulus (e.g., via therapy generation circuitry 34, alone or in combination with processing circuitry 30) to elicit the evoked potential. For example, endovascular device 16 can generate and deliver electrical stimulation to tissue in brain 18 and sense an evoked compound action potential (ECAP). An ECAP is synchronous firing of a population of neurons which occurs in response to the application of a stimulus including, in some cases, an electrical stimulus by endovascular device 16. The ECAP may be detectable as being a separate event from the stimulus itself, and the ECAP may reveal characteristics of the effect of the stimulus on the tissue.

In some examples, sensing circuitry 36 and/or processing circuitry 30 includes signal processing circuitry configured to perform any suitable analog conditioning of the sensed physiological signals. For example, sensing circuitry 36 may communicate to processing circuitry 30 an unaltered (e.g., raw) signal. Processing circuitry 30 may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the conditioned analog signals are processed by an analog-to-digital converter of processing circuitry 30 or other component to convert the conditioned analog signals into digital signals. In some examples, processing circuitry 30 operates on the analog or digital form of the signals to separate out different components of the signals. In some examples, sensing circuitry 36 and/or processing circuitry 30 performs any suitable digital conditioning of the converted digital signals, such as low pass, high pass, band pass, notch, averaging, or any other suitable filtering, amplifying, performing an operation on the signal, performing any other suitable digital conditioning, or any combination thereof. Additionally or alternatively, sensing circuitry 36 may include signal processing circuitry to modify one or more raw signals and communicate to processing circuitry 30 one or more modified signals.

In some examples, processing circuitry 30, alone or in combination with therapy generation circuitry 34 and/or sensing circuitry 36, is configured to operate therapy device 14 (including electrodes 17, endovascular device 16, etc.) in a trial mode for a trial period to determine an efficacy of electrical stimulation or sensing. As described above, a trial mode can include a trial period of stimulation and/or sensing to determine, evaluate, or confirm an efficacy of stimulation and/or sensing. In some examples, processing circuitry 30, alone or in combination with therapy generation circuitry 34 and/or sensing circuitry 36, is configured to deliver electrical stimulation therapy and/or sense a patient parameter during the trial period. In some examples, processing circuitry 30 is configured to determine, evaluate, or confirm an efficacy of stimulation and/or sensing. For example, processing circuitry 30 may determine one or more therapy parameters for chronic stimulation and/or sensing based on the trial period.

Although shown as part of therapy device 14 in FIG. 1B, in other examples, sensing circuitry 36 is part of a device separate from therapy device 14. For example, sensing circuitry 36 can be part of an implantable sensing device implanted in patient 12.

Processing circuitry 30, as well as other processors, processing circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 30 includes multiple components, such as any combination of one or more microprocessors, one or more DSPs, one or more ASICs, or one or more FPGAs, as well as other discrete or integrated logic circuitry, and/or analog circuitry.

Memory 32 is configured to store program instructions, such as software, which may include one or more program modules, which are executable by processing circuitry 30. When executed by processing circuitry 30, such program instructions may cause processing circuitry 30 to provide the functionality ascribed to processing circuitry 30 herein. The program instructions may be embodied in software and/or firmware. Memory 32 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processing circuitry 30 is configured to control telemetry circuitry 38 to send and receive information. Telemetry circuitry 38, as well as telemetry modules in other devices described herein, such as programmer 20 (FIG. 1A), may accomplish communication by any suitable communication techniques, such as RF communication techniques. In addition, telemetry circuitry 38 may communicate with external medical device programmer 20 via proximal inductive interaction of therapy device 14 with programmer 20. Accordingly, telemetry circuitry 38 may send information to external programmer 20 on a continuous basis, at periodic intervals, or upon request from therapy device 14 or programmer 20.

Power source 40 is configured to deliver operating power to various components of therapy device 14. Power source 40 may include a small rechargeable or non-rechargeable battery and a power generation circuit to produce the operating power. Recharging may be accomplished through proximal inductive interaction between an external charger and an inductive charging coil within therapy device 14. In some examples, power requirements may be small enough to allow therapy device 14 to utilize patient motion and implement a kinetic energy-scavenging device to trickle charge a rechargeable battery. In other examples, traditional batteries may be used for a limited period of time.

In some examples, endovascular device 16 is configured to be a standalone electrical stimulation device and can include one or more elements of therapy device 14 shown in FIG. 1B.

In some examples of medical devices described herein, a medical device includes an expandable structure with a variable size. For example, rather than use an existing expandable structure, such as a stent, an endovascular device may use a flexible template as an expandable structure that can be configured with various shapes and provides a large surface area with which to adhere to a flexible polymer packaging for coupling the electrodes. Additionally, such endovascular devices may be manufactured using planar manufacturing techniques that enable large scale, parallel manufacturing, and precise placement of electrodes.

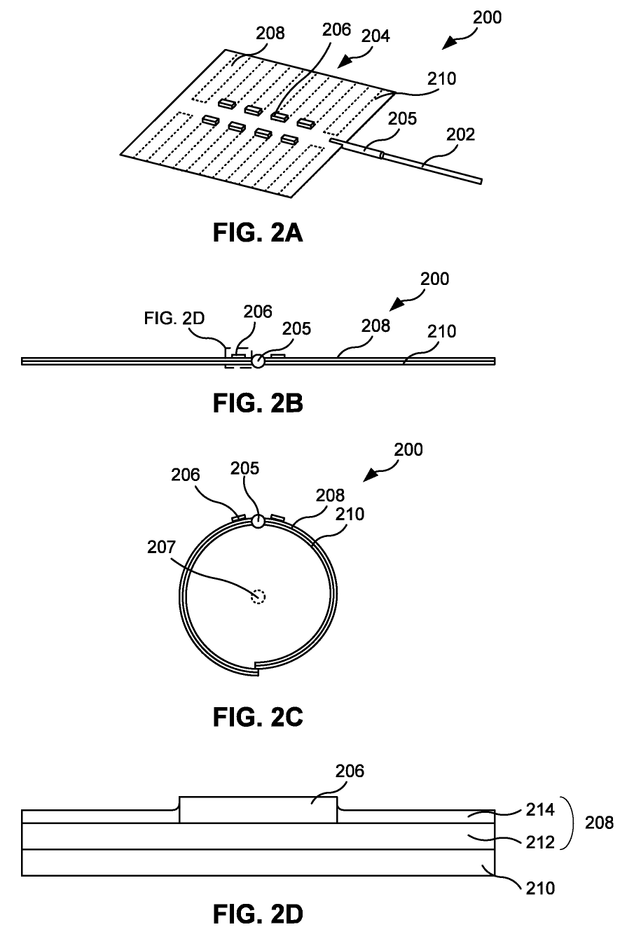

FIG. 2A is a perspective view of a distal portion of an example medical device 200, such as an endovascular device, in a pre-delivery configuration, while FIG. 2B is a cross-sectional axis view diagram of the example medical device 200 of FIG. 2A in the pre-delivery configuration. Medical device 200 includes an elongated body 202, an electrode array 204 including one or more electrodes 206 and a flexible polymer packaging 208, and an expandable structure 210. Unless otherwise specified, elongated body 202, electrodes 206, flexible polymer packaging 208, and expandable structure 210 may be consistent with a general description of elongated body 23, electrodes 17, flexible polymer packaging 22, and expandable structure 19 of FIGS. 1A and 1B.

Elongated body 202 is configured to be introduced into a patient, such as into a blood vessel or beneath skin, and remain at least partially in the patient (e.g., a distal portion of elongated body 202). Elongated body 202 may also include a connector 205 configured to mechanically couple to flexible polymer packaging 208.

In the examples of FIGS. 2A and 2B, medical device 200 includes a non-tubular expandable structure 210. A non-tubular expandable structure may include any expandable structure that does not form a continuous tube. For example, a non-tubular expandable structure may include a cylinder formed from a planar sheet in which ends of the planar sheet are not secured to each other, thereby forming a discontinuity. As will be described further below, such discontinuity may enable a variable diameter for various delivery configurations, including planar configurations (e.g., for subcutaneous placement). Non-tubular expandable structure 210 may be configured to secure a distal end of medical device 200 in the blood vessel of the patient, such that expandable structure 210 may substantially remain at a particular position despite movement of the patient.

In some examples, expandable structure 210 is a flexible metal template. For example, expandable structure 210 may be a substantially two-dimensional sheet having a bendability that enables expandable structure 210 to be collapsible into a delivery sheath, deployable from the delivery sheath, and forceful for exerting a force against the inner surface of the blood vessel to secure electrodes 206 against the inner surface of the blood vessel. In the example of FIG. 2A, expandable structure 210 includes a plurality of projections extending from a central spine. Such projections may cumulatively exert a force sufficient to secure expandable structure 210 in the blood vessel while enabling bendability of expandable structure 210.

In some examples, elongated body 202 is mechanically coupled to the expandable structure 210. In the example of FIG. 2A, elongated body 202 may be mechanically coupled to expandable structure 210 at the central spine. In other examples, elongated body 202 may only be mechanically coupled to electrode array 204, such as to permit additional flexibility and strain relief between elongated body 202 and expandable structure 210.

Electrode array 204 includes flexible polymer packaging 208 overlying at least a portion of expandable structure 210. In the example of FIGS. 2A and 2B, flexible polymer packaging 208 overlies an entirety of expandable structure 210, including portions that are not directly supported by expandable structure 210. However, in other examples, flexible polymer packaging 208 may only cover less than all of expandable structure 210. Regardless of an extent of coverage, flexible polymer packaging 208 may have a relatively high amount of surface area that is in contact with expandable structure 210, such as at least twenty percent (%) of a total outer surface area of expandable structure 210. As a result, flexible polymer packaging 208 may resist peeling or delamination forces, and remain coupled to expandable structure 210.

Medical device 200 includes an electrode array 204 coupled to expandable structure 210. Electrode array 204 includes one or more electrodes 206 electrically coupled to elongated body 202, such that electrodes 206 may receive therapy delivery signals to elongated body 202 and/or send sensing signals to elongated body 202. Electrodes 206 are also mechanically coupled to flexible polymer packaging 208. For example, electrodes 206 may be adhered to and/or embedded in an outer surface of flexible polymer packaging, either directly or through an adhesive. During operation, electrodes 206 may contact an inner surface of the blood vessel to deliver electrical stimulation to tissue of the patient or sense a patient parameter of the tissue of the patient from a location within the blood vessel.

Expandable structure 210 is configured to expand and contract between a pre-delivery configuration and a delivery configuration. In the example of FIGS. 2A and 2B, expandable structure 210 is a planar stent preform configured to bend from a planar form into a cylindrical form. In some examples, the planar form may be associated with the pre-delivery configuration prior to delivering medical device 200 through the blood vessel, while the cylindrical form may be associated with a delivery configuration while delivering medical device 200 through vasculature or a deployed configuration once medical device 200 is positioned within the vasculature. For example, the cylindrical form may be configured for positioning with a blood vessel. In other examples, the planar form may be associated with both a pre-delivery configuration and a deployed configuration, while the cylindrical form may only be associated with a delivery configuration. For example, the planar form may be configured for positioning subcutaneously, in which a final planar configuration may be desired.

FIG. 2C is a cross-sectional axis view diagram of the example medical device 200 of FIG. 2A in a delivery configuration. Expandable structure 210 is configured to bend around a longitudinal axis 207, which may represent an average radial center of expandable structure 210 in the delivery configuration. A diameter of expandable structure 210 in the delivery configuration may vary depending on an extent of bending around longitudinal axis 207, such that expandable structure 210 may accommodate different sizes of vessels. For example, ends of medical device 200 may overlap, such as shown in FIGS. 2C, and an amount of the overlap may increase as a diameter of expandable structure 210 decreases. Once deployed, expandable structure 210 may expand to contact an inner surface of the vessel.

In some examples, expandable structure 210 may be configured to self-deploy, such as self-expand, such as in response to removal of a constraint (e.g., a delivery sheath), until an outer surface of medical device 200 contacts the inner surface. For example, expandable structure 210 may be configured to self-expand from a delivery configuration to a deployed configuration to position the one or more electrodes to deliver electrical stimulation to tissue of the patient or sense a patient parameter from a location within the blood vessel. Expandable structure 210 may continue to exert an outward radial force to assist in holding electrodes 206 in contact with the inner surface of the blood vessel.

In some examples, expandable structure 210 may be configured to selectively expand in response to a stimulus, such as in response to a temperature or electrical stimulus, until an outer surface of medical device 200 contacts the inner surface. For example, expandable structure 210 may include a shape memory metal having a first state in the delivery configuration and a second state in the deployed configuration, and in which transition from the first state to the second state may be in response to an increase in temperature caused by resistive heating.

In the example of FIG. 2C, expandable structure 210 is configured to bend such that electrode array 204 faces radially outward. For example, expandable structure 210 may be positioned within a vessel, in which therapy may be delivered radially outward to tissues at or beyond the blood vessel. However, in other examples, expandable structure 210 may be configured to bend such that electrode array 204 faces radially inward. For example, medical device 200 may be a cuff endovascular device that is configured to fit around a structure and deliver therapy radially inward.

Flexible polymer packaging 208 may assist in accommodating expansion and/or contraction of expandable structure 210 while still securing and accurately positioning electrodes 206. FIG. 2D is an expanded cross-sectional axis view diagram of the example endovascular device of FIG. 2A. Flexible polymer packaging 208 may include different functional layers, including a support layer 212 and an encapsulation layer 214 to aid in securing electrodes 206 to expandable structure 210.

Support layer 212 is coupled to expandable structure 210 and configured to secure flexible polymer packaging 208, and therefore electrodes 206, to expandable structure 210. For example, support layer 212 may share a large surface area with expandable structure 210, such that support layer 212 may adhere to expandable structure 210. In some examples, support layer 212 may be relatively flexible, such that bending of expandable structure 210 may cause expansion or contraction of support layer 212 without support layer 212 becoming delaminated from expandable structure 210.

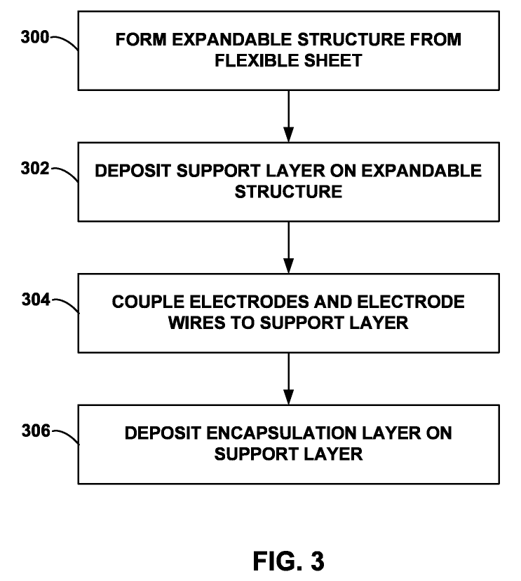

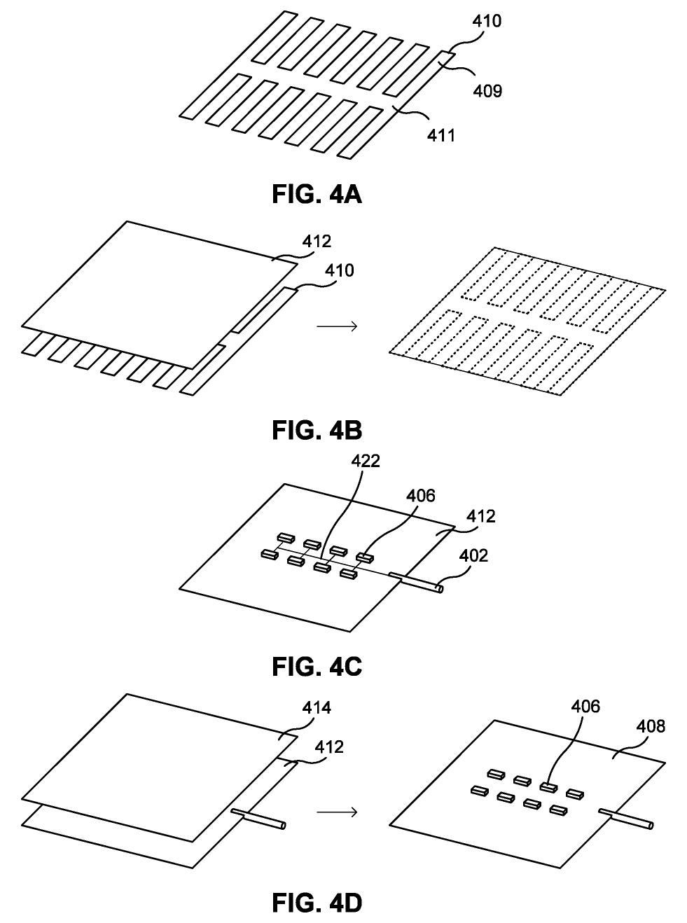

Encapsulation layer 214 overlies support layer 212. Encapsulation layer 214 may be configured to encapsulate and/or secure various components of electrode array 204, including electrodes 206, electrode wires (not shown), or other components that may require insulation and/or be more securely attached by an additional layer. As will be described in FIGS. 3 and 4A-4D, electrodes 206 may be attached to expandable structure 210 after support layer 212 has been deposited on expandable structure 210, such that electrodes 206 may only be adhered by a single surface and/or components such as wires may be uninsulated. Encapsulation layer 214 may be configured to encapsulate the electrode wires between electrodes 206 and elongated body 202. In some examples, electrodes 206 may be embedded in encapsulation layer 214. For example, in FIG. 2D, encapsulation layer 214 contacts at least a portion of sides of electrode 206, such as greater than 50% of a surface area of lateral sides of electrodes 206. Such contact may assist in securing electrodes 206 to flexible polymer packaging 208.

Each of support layer 212 and encapsulation layer 214 may include a biocompatible polymer. The biocompatible polymers may be selected for properties including, but not limited to, biocompatibility (e.g., non-toxic, non-immunogenic, and non-carcinogenic); flexibility and elasticity (e.g., to accommodate movement and deformation without breaking or losing its structural integrity); mechanical strength (e.g., sufficient tensile strength and durability to withstand physiological conditions and mechanical stresses caused by bending); surface properties (e.g., increasing adhesion, reducing thrombogenicity, and increasing surface compatibility); thermal stability (e.g., remain stable and maintain its properties at body temperature (37°C), and optionally, during sterilization processes); chemical stability (e.g., resist degradation or alteration when exposed to bodily fluids and other chemicals it might encounter in the medical environment), ease of fabrication (e.g., amenable to various processing techniques, such as molding and extrusion); and the like. A variety of biocompatible polymers may be used for support layer 212 including, but not limited to, polyurethanes (PU), polydimethylsiloxane (PDMS, i.e., silicone), polyethylene glycol (PEG), polylactic acid (PLA), polyglycolic acid (PGA), polycaprolactone (PCL), polytetrafluoroethylene (PTFE), polyvinyl alcohol (PVA), poly(L-lactic-co-glycolic acid) (PLGA), and the like.

While medical device 200 of FIGS. 2A-2D have been described with respect to an endovascular device, in some examples, medical device 200 may be a subcutaneous device configured to deliver therapy and/or send signals to tissues under skin of the patient. For example, medical device 200 may be configured to self-expand from a delivery configuration to a deployed configuration to position the one or more electrodes to deliver electrical stimulation to tissue of the patient or sense a patient parameter from a location beneath skin of the patient. Medical device 200 may be delivered through a needle or other delivery device.

FIG. 3 is a flowchart of an example method for fabricating a medical device, such as an endovascular device. The method of FIG. 3 will be described with respect to FIGS. 4A-4D, but may be used to form other medical devices. Unless otherwise noted, components of FIGS. 4A-4D may be operably and/or structurally similar to similarly numbered components of FIGS. 2A-2D.

In some examples, the method includes forming expandable structure 410 from a flexible metal template (300). FIG. 4A is a perspective view of a flexible metal template used as expandable structure 410 for forming an example endovascular device. The flexible metal template may be machined to form a central spine 411 and a plurality of projections 409 extending from central spine 411.

The method of FIG. 3 includes forming a flexible polymer packaging 408 on at least a portion of expandable structure 410. In examples in which flexible polymer packaging 408 includes two or more functional layers, the method of FIG. 3 includes depositing a support layer 412 on expandable structure 410 (302). FIG. 4B is a perspective views of support layer 412 overlying the flexible metal template of FIG. 4A, both prior to and after deposition. Support layer 412 may be a rigid or semi-rigid sheet configured to be positioned over expandable structure 410. Support layer 412 may be adhered to expandable structure 410 directly (e.g., via localized melting) or indirectly via an adhesive. In other examples support structure 412 may be applied as a fluid, such as through extrusion.

The method of FIG. 3 includes coupling electrodes 406 and electrode wires 422 to support layer 412 (304). FIG. 4C is a perspective view diagram of a plurality of electrodes 406 overlying support layer 412 of FIG. 4B. Elongated body 402 may be mechanically coupled to flexible polymer packaging 408. In some examples, elongated body 402 may also be mechanically coupled to expandable structure 410, such as via crimping or welding. To couple electrodes 406 to support layer 412, electrodes 406 may be positioned and adhered at various positions on support layer 412 that result in positioning at various axial and circumferential positions on flexible polymer packaging 408 once expandable structure 410 has been bend around an axis. Electrode wires 422 extend from electrodes 406 to elongated body 402 to electrically couple electrodes 406 to elongated body 402 and deliver signals to and/or receive signals from elongated body 402.

The method of FIG. 3 includes depositing encapsulation layer 414 on support layer 412 (306). FIG. 4D is a perspective view diagram of an encapsulation layer overlying the support layer of FIG. 4C, both prior to and after deposition. While shown as being deposited on an entirety of support layer 412, in some examples, encapsulation layer 414 may only be deposited on particular portions of support layer 412, such as portions underlying electrode wires 422 and/or adjacent to electrodes 406. In some examples, encapsulation layer 414 may be deposited over electrodes 406 as a rigid or semi-rigid layer, and various openings may be formed in encapsulation layer 414, such as through cutting or localized melting. In other examples, openings may be included in encapsulation layer 414, thereby exposing surfaces of electrodes 406. In some examples, encapsulation layer 414 may be applied as fluid, such as through extrusion or molding, such that encapsulation layer 414 may flow over support layer 412 and electrode wires 422 and around electrodes 406. Encapsulation layer 414 encapsulates electrode wires 422. In some examples, encapsulation layer 414 may also embed electrodes 406, such as by contacting lateral surfaces of electrodes 406.

In some examples of medical devices described herein, a medical device includes an expandable structure with a range of sizes. For example, an endovascular device may use an existing expandable structure, such as a stent, that can be embedded into the flexible polymer packaging to position the electrodes on the expandable structure.

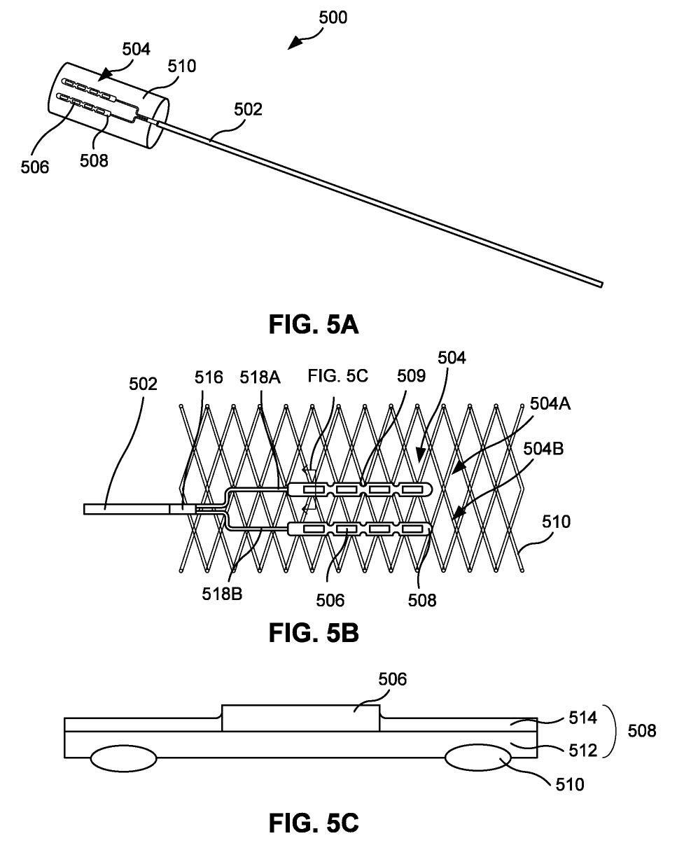

FIG. 5A is a perspective view of a distal portion of an example endovascular device 500, while FIG. 5B is a top view of the example endovascular device 500 of FIG. 5A. Endovascular device 500 includes elongated body 502, electrode array 504 including one or more electrodes 506 and a flexible polymer packaging 508, and an expandable structure 510. Unless otherwise specified, elongated body 502, electrodes 506, flexible polymer packaging 508, and expandable structure 510 may be consistent with a general description of elongated body 23, electrodes 17, flexible polymer packaging 22, and expandable structure 19 of FIGS. 1A and 1B.

Elongated body 502 is configured to be introduced into a blood vessel of a patient and remain at least partially in the blood vessel of the patient (e.g., a distal portion of elongated body 502). Elongated body 502 also includes array wires 518A, 518B configured to electrically couple to electrodes 506 and mechanically couple to flexible polymer packaging 508.

In the example of FIGS. 5A and 5B, endovascular device 500 includes a tubular expandable structure 510. A tubular expandable structure may include any expandable structure that forms a structurally continuous tube, such as a stent. Expandable structure 510 is configured to expand from a delivery configuration to a deployed configuration to position electrodes 506 to deliver electrical stimulation to tissue of the patient or sense a patient parameter from a location within the blood vessel. Expandable structure 510 may be configured to secure a distal end of endovascular device 500 in the blood vessel of the patient, such that expandable structure 510 may substantially remain at a particular position despite movement of the patient. In some examples, expandable structure 510 is a tubular stent that includes a plurality of interconnected struts.

Elongated body 502 is mechanically coupled to the expandable structure 510 via connector 516. Connector 516 may be a ring or other structure configured to be crimped or welded to one or more structures of expandable structure 510. In examples in which expandable structure 510 is a stent, connector 516 may be mechanically coupled to one or more struts of expandable structure 510.

Endovascular device 500 includes an electrode array 504 coupled to expandable structure 510. Each electrode array 504 includes flexible polymer packaging 508 overlying at least a portion of expandable structure 510. In some examples, flexible polymer packaging 508 may be molded around at least a portion of expandable structure 510, such that expandable structure 510 is embedded within flexible polymer packaging 508. Such molding may improve adhesion between expandable structure 510 and flexible polymer packaging 508, particularly during expansion or contraction of expandable structure 510. As a result, flexible polymer packaging 508 may resist peeling or delamination forces, and remain coupled to expandable structure 510.

Electrode array 504 includes one or more electrodes 506 electrically coupled to elongated body 502, such that electrodes 506 may receive therapy delivery signals to elongated body 502 and/or send sensing signals to elongated body 502. Electrodes 506 are also coupled to flexible polymer packaging 508. For example, electrodes 506 may be adhered to and/or embedded in an outer surface of flexible polymer packaging, either directly or through an adhesive. During operation, electrodes 506 may contact an inner surface of the blood vessel to deliver electrical stimulation to tissue of the patient or sense a patient parameter from a location within the blood vessel.

Expandable structure 510 is configured to expand and contract between a delivery configuration and a deployed configuration. To maintain a relatively constant circumferential position of electrodes 506 around expandable structure 510, flexible polymer packaging 508 may be configured to expand and contract with expandable structure. Such expansion and contraction may result from compositional properties, such as flexibility or elasticity of the polymer, of flexible polymer packaging and/or structural features, such as accommodating structures, of electrode array 504.

In some examples, electrode array 504 includes two or more groups of electrodes 506, such as may each be positioned at a particular circumferential position around expandable structure 510. In the example of FIGS. 5A and 5B, electrode array 504 includes a first electrode array 504A and a second electrode array 504B positioned around expandable structure 510 at different longitudinal positions. First electrode array 504A includes a first portion of flexible polymer packaging 508 and a first portion of electrodes 506 coupled to the first portion of the flexible polymer packaging 508 and electrically coupled to the elongated body 502 via a first array wire 518A. Second electrode array 504B includes a second portion of flexible polymer packaging 508 and a second portion of electrodes 506 coupled to the first portion of the flexible polymer packaging 508 and electrically coupled to the elongated body 502 via a second array wire 518B.

Electrode array 504 may be configured to permit relative movement between first electrode array 504A and second electrode array 504B in response to radial or axial expansion or contraction of expandable structure 510. To maintain the circumferential position of each electrode array 504A, 504B, and/or to reduce bunching with such expansion or contraction, each electrode array 504A, 504B may be configured to separate when expandable structure 510 expands radially outward and move together when expandable structure 510 contracts radially inward. Array wires 518A, 518B may be sufficiently long to accommodate movement through a desired range of radial expansion and contraction of expandable structure 510. In some examples, different portions of electrodes 506 may have different properties. For example, the first portion of electrodes 506 and the second portion of electrodes 506 may have different polarities. In such examples, first and second electrode arrays 504A, 504B may be separated by a particular spacing, such as from about three to about 7 millimeters. In the example of FIG. 5B, each electrode array 504A, 504B may include cutouts 509 positioned between adjacent electrodes. Such cutouts 509 may be configured to aid in axial expansion and/or contraction of electrode arrays 504A, 504B.

FIG. 5C is an expanded cross-sectional axis view diagram of the example endovascular device of FIG. 5A. Flexible polymer packaging 508 may include different functional layers, including a support layer 512 and an encapsulation layer 514. Each of support layer 512 and encapsulation layer 514 may include a biocompatible polymer, such as biocompatible polymers described for support layer 212 and encapsulation layer 214 of FIG. 2D above.

Support layer 512 is coupled to a portion of expandable structure 510 and configured to secure flexible polymer packaging 508, and therefore electrodes 506, to expandable structure 510. For example, support layer 512 may partially or fully embed portions of expandable structure 510, such that support layer 512 may adhere to expandable structure 510. In the example of FIG. 5C, struts of expandable structure 510 is partially embedded in support layer 512; however, in other examples, struts of expandable structure 510 may be fully embedded in support layer 512, or embedded between support layer 512 and encapsulation layer 514. In some examples, support layer 512 may be relatively flexible, such that radial and/or axial expansion and contraction of expandable structure 510 may cause expansion or contraction of support layer 512 without support layer 512 becoming detached from expandable structure 510.

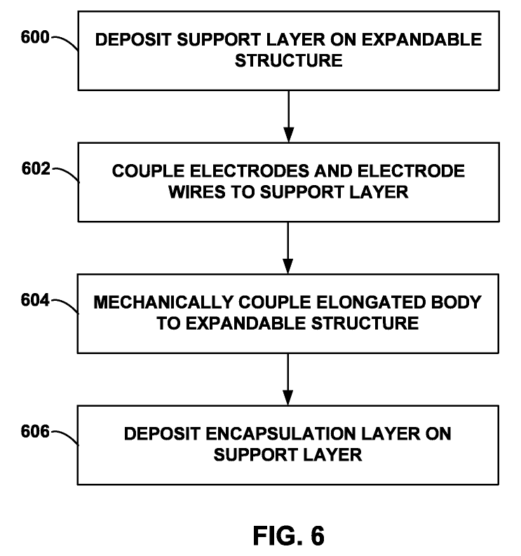

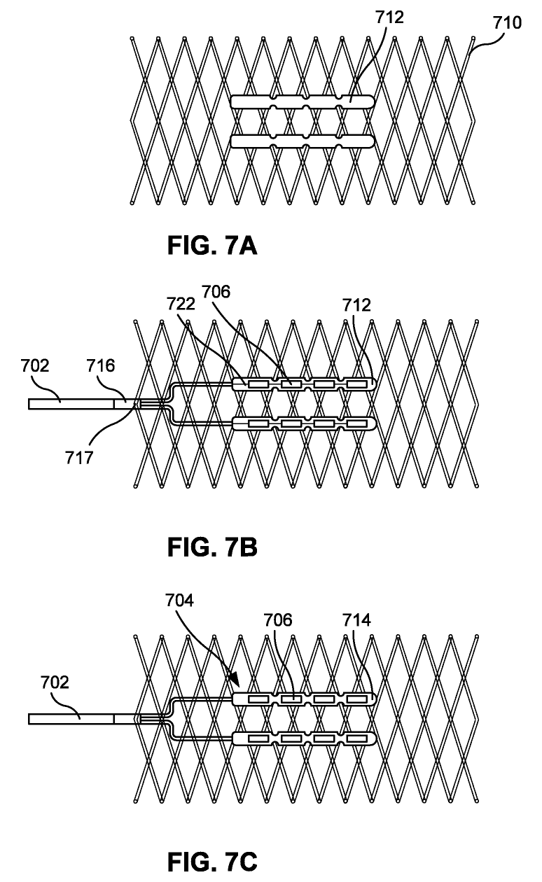

Encapsulation layer 514 overlies support layer 512. Encapsulation layer 514 may be configured to encapsulate and/or secure various components of electrode array 504, including electrodes 506, electrode wires (not shown), or other components that may require insulation and/or be more securely attached by an additional layer. As will be described in FIGS. 6 and 7A-7C, electrodes 506 may be coupled to expandable structure 510 after support layer 512 has been deposited on expandable structure 510, such that electrodes 506 may only be adhered by a single surface and/or components such as wires may be uninsulated. Encapsulation layer 514 may be configured to encapsulate the electrode wires between electrodes 506 and array wires 518. In some examples, electrodes 506 may be embedded in encapsulation layer 514. For example, in FIG. 5C, encapsulation layer 514 contacts at least a portion of sides of electrode 506. Such contact may assist in securing electrodes 506 to flexible polymer packaging 508.

FIG. 6 is a flowchart of an example method for fabricating an endovascular device. The method of FIG. 6 will be described with respect to FIGS. 7A-7C, but may be used to form other endovascular devices. Unless otherwise noted, components of FIGS. 7A-7C may be operably and/or structurally similar to similarly numbered components of FIGS. 5A-5C.

The method of FIG. 6 includes molding a flexible polymer packaging 708 on at least a portion of expandable structure 710. In examples in which flexible polymer packaging 708 includes two or more layers, the method of FIG. 6 includes depositing support layer 712 on expandable structure 710 (600). FIG. 7A is a top view of a support layer overlying a tubular expandable structure. In some examples, support layer 712 may be formed by extruding a biocompatible polymer onto expandable structure 710, such that support layer 712 molds around and embeds at least a portion of expandable structure 710. For example, in endovascular devices in which expandable structure 710 is a tubular stent that includes a plurality of interconnected struts, support layer 712 may flow around at least a portion of the plurality of interconnected struts to embed the struts in support layer 712. In other examples, support layer 712 may be a rigid or semi-rigid sheet configured to be positioned over or under expandable structure 710. Support layer 712 may be adhered to expandable structure 710 directly (e.g., via localized melting), indirectly via an adhesive, or against encapsulation layer 714 (e.g., such that expandable structure 710 is sandwiched between support layer 712 and encapsulation layer 714.

The method of FIG. 6 includes coupling electrodes 706 and electrode wires 722 to support layer 712 (602). FIG. 7B is a top view of a plurality of electrodes 706 overlying support layer 712 of FIG. 7A. Electrodes 706 may be positioned at various axial and circumferential positions on flexible polymer packaging 708 corresponding to desired axial and circumferential positions around expandable structure 710. Electrode wires 722 extend from electrodes 706 to elongated body 702 via array wires 718 to deliver signals to and/or receive signals from lead 702.

The method of FIG. 6 includes mechanically coupling elongated body 702 to expandable structure 710 (604). For example, lead connector 716 may be welded to one or more structures of expandable structure 710. In some examples, elongated body 702 may also be mechanically coupled to flexible polymer packaging 708, such as via array wires 718.

The method of FIG. 6 includes depositing encapsulation layer 714 on support layer 712 (606). FIG. 7C is a top view of an encapsulation layer 714 overlying support layer 712 of FIG. 7B. In some examples, encapsulation layer 714 may be deposited over electrodes 706, and various openings may be formed in encapsulation layer 714, such as through cutting or localized melting. In other examples, openings may be included in encapsulation layer 714, thereby exposing surfaces of electrodes 706. In some examples, encapsulation layer 714 is a fluid that is extruded onto support layer 712, thereby covering electrode wires 722 and flow around electrodes 706. Encapsulation layer 714 encapsulates electrode wires 722. In some examples, encapsulation layer 714 may also embed electrodes 706, such as by contacting lateral surfaces of electrodes 706.

In some examples, forming flexible polymer packaging 708 may include forming two separate electrode arrays. For example, electrode array 704 may include two separate electrode arrays, such that forming flexible polymer packaging 708 may include depositing two discrete portions of support layer 712, positioning two different groups of electrodes 706 on support layer 712, and depositing two discrete portions of encapsulation layer 714 on support layer 712. The electrode arrays may be separated by a particular spacing, such as less than about 1 millimeter.

In some examples of medical devices described herein, a medical device includes an expandable structure configured to be positioned within or around an existing expandable structure within a vessel, such as a stent. For example, an endovascular device may use an expandable structure that is coupled to the flexible polymer packaging to position the electrodes within the existing expandable structure.

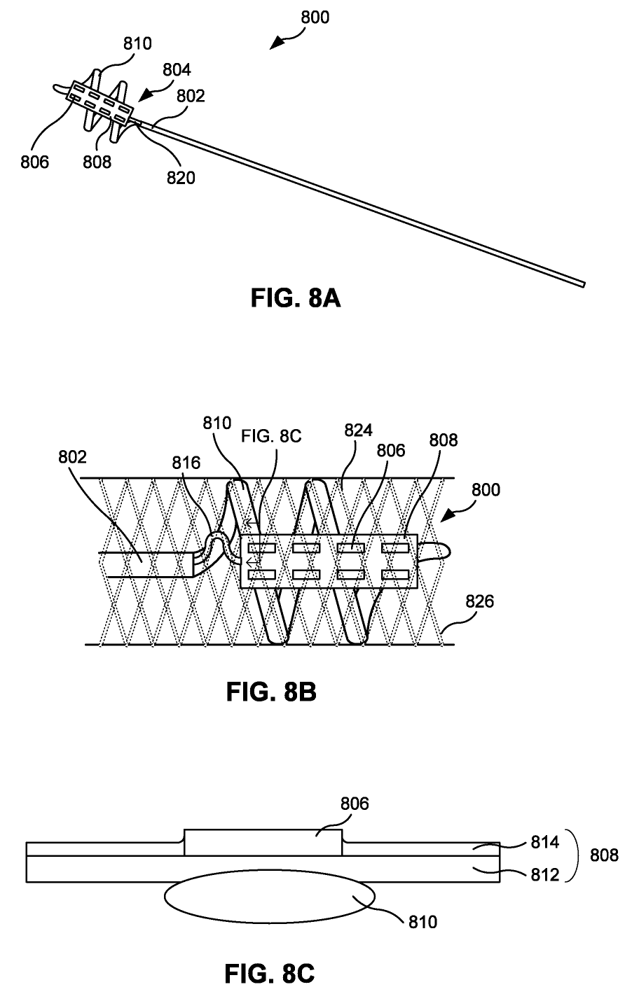

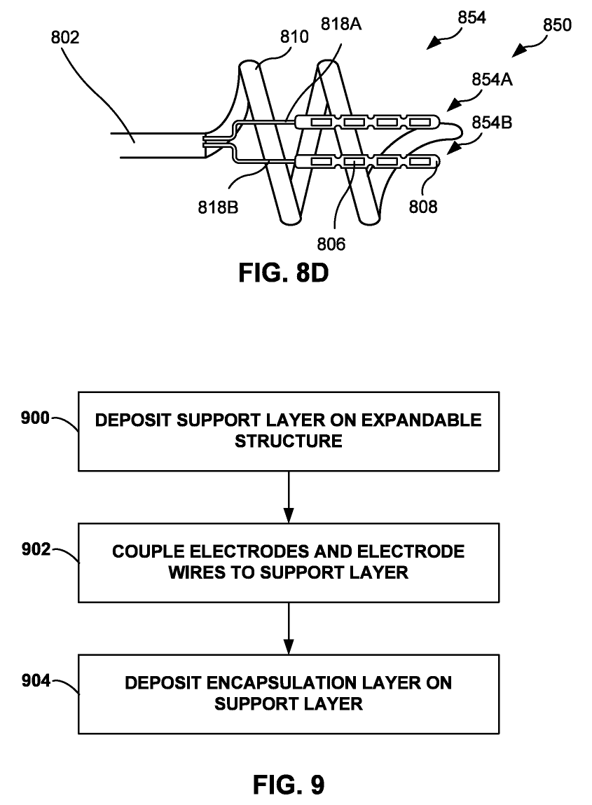

FIG. 8A is a perspective view of a distal portion of an example endovascular device 800, while FIG. 8B is a side view of the endovascular device 800 of FIG. 8A positioned within a stent. Endovascular device 800 includes an elongated body 802, an electrode array 804 including one or more electrodes 806 and a flexible polymer packaging 808, and an expandable structure 810. Unless otherwise specified, elongated body 802, electrodes 806, flexible polymer packaging 808, and expandable structure 810 may be consistent with a general description of elongated body 23, electrodes 17, flexible polymer packaging 22, and expandable structure 19 of FIGS. 1A and 1B.

Elongated body 802 is configured to be introduced into a blood vessel of a patient and remain at least partially in the blood vessel of the patient (e.g., a distal portion of elongated body 802). Elongated body 802 also includes array wires 518A, 518B configured to mechanically couple to flexible polymer packaging 808.

Expandable structure 810 is configured to expand from a delivery configuration to a deployed configuration within a stent 826 to position electrodes 806 to deliver electrical stimulation to tissue of the patient or sense a patient parameter from a location within the blood vessel and existing expandable structure within the blood vessel. Expandable structure 810 may be configured to secure a distal end of endovascular device 800 in stent 826 in the blood vessel of the patient, such that expandable structure 810 may substantially remain at a particular position despite movement of the patient. In the example, of FIG. 8A, endovascular device 800 includes a helical expandable structure 810. Helical expandable structure 810 may be configured to expand from a relatively straight delivery configuration to a helical deployed configuration within stent 826. Elongated body 802 is mechanically coupled to the expandable structure 810. While expandable structure 810 has been described as being positioned within stent 826, in some instances, expandable structure 810 may be positioned around stent 826.

Endovascular device 800 includes an electrode array 804 coupled to expandable structure 810. Electrode array 804 includes flexible polymer packaging 808 overlying at least a portion of expandable structure 810. In some examples, flexible polymer packaging 808 may be molded around at least a portion of expandable structure 810, such that expandable structure 810 may be embedded within flexible polymer packaging 808. Such molding may improve adhesion between expandable structure 810 and flexible polymer packaging 808, particularly during expansion or contraction of expandable structure 810. As a result, flexible polymer packaging 808 may resist peeling or delamination forces, and remain coupled to expandable structure 810.

Electrode array 804 includes one or more electrodes 806 electrically coupled to elongated body 802, such that electrodes 806 may receive therapy delivery signals to lead 802 and/or send sensing signals to lead 802. Electrodes 806 are also coupled to flexible polymer packaging 808. For example, electrodes 806 may be adhered to and/or embedded in an outer surface of flexible polymer packaging 808, either directly or through an adhesive. During operation, electrodes 806 may contact an inner surface of the blood vessel to deliver electrical stimulation to tissue of the patient or sense a patient parameter from a location within the blood vessel.

In the example of FIGS. 8A and 8B, flexible polymer packaging 808 is configured to expand and contract in response to expansion and contraction of expandable structure 810. For example, flexible polymer packaging 808 may be configured to stretch circumferentially and/or longitudinally to accommodate radial or longitudinal expansion of expandable device 810 and/or stent 826 in which electrode array 804 is positioned. For example, flexible polymer packaging 808 may be configured to expand at least ten percent along and around the longitudinal axis of stent 826. In some examples, expandable structure 810 further includes a strain relief 816. Strain relief 816 is configured to expand along a longitudinal axis in response to expansion or contraction of expandable structure 810 and/or stent 826. In some examples, flexible polymer packaging 808 is configured to conform to a surface of stent 826. For example, an outer surface of flexible polymer packaging 808 may be configured to conform to struts of stent 826, such that electrodes 806 may contact the inner surface of the blood vessel.

FIG. 8C is an expanded cross-sectional axis view diagram of the example endovascular device of FIG. 8B. Flexible polymer packaging 808 may include different functional layers, including a support layer 812 and an encapsulation layer 814. Each of support layer 812 and encapsulation layer 814 may include a biocompatible polymer, such as biocompatible polymers described for support layer 212 and encapsulation layer 214 of FIG. 2D above.

Support layer 812 is coupled to a portion of expandable structure 810 and configured to secure flexible polymer packaging 808, and therefore electrodes 806, to expandable structure 810. For example, support layer 812 may partially or fully embed portions of expandable structure 810, such that support layer 812 may adhere to expandable structure 810. In the example of FIG. 8C, a body of helical expandable structure 810 is partially embedded in support layer 812; however, in other examples, the body of expandable structure 810 may be fully embedded in support layer 812, or embedded between support layer 812 and encapsulation layer 814.

In some examples, support layer 812 may be relatively flexible, such that radial and/or axial expansion and contraction of expandable structure 810 may cause expansion or contraction of support layer 812 without support layer 812 becoming detached from expandable structure 810. In some examples, support layer 812 may also be relatively supportive, particularly if expandable structure 810 does not include a large number of support structures, such as struts.

Encapsulation layer 814 overlies support layer 812. Encapsulation layer 814 may be configured to encapsulate and/or secure various components of electrode array 804, including electrodes 806, electrode wires (not shown), or other components that may require insulation and/or be more securely attached by an additional layer. Encapsulation layer 814 may be configured to encapsulate the electrode wires between electrodes 806 and array wires 818. In some examples, electrodes 806 may be embedded in encapsulation layer 814. For example, in FIG. 8C, encapsulation layer 814 contacts at least a portion of sides of electrode 806.

FIG. 8D is a side view of an endovascular device 850 that includes an alternative electrode array 854. Endovascular device 850 includes elongated body 802, electrode array 854 including one or more electrodes 806 and a flexible polymer packaging 808, and expandable structure 810. Electrode array 854 may be similar in structure and operation to electrode array 504 of FIGS. 5A and 5B. For example, electrode array 854 includes two or more groups of electrodes 856, such as may each be positioned at a particular circumferential position around expandable structure 810 and configured to expand and contract in response to expansion or contraction of expandable structure 810. In the example of FIG. 8D, electrode array 854 includes a first electrode array 854A and a second electrode array 854B, which may be similar to first electrode array 504A and second electrode array 504B. However, flexible polymer packaging 808 of each electrode array 854A, 854B may be firmer, such that each electrode array 854A, 854B may maintain a shape.

FIG. 9 is a flowchart of an example method for fabricating an endovascular device. The method of FIG. 9 will be described with respect to FIGS. 8A–8C, but may be used to form other endovascular devices. The method of FIG. 9 includes forming a flexible polymer packaging 808 on at least a portion of expandable structure 810. In examples in which flexible polymer packaging 808 includes two or more layers, the method of FIG. 9 includes depositing support layer 812 on expandable structure 810 (900). In some examples, support layer 812 may be a rigid or semi-rigid sheet configured to be positioned over or under expandable structure 810. Support layer 812 may be adhered to expandable structure 810 directly (e.g., via localized melting), indirectly via an adhesive, or against encapsulation layer 814 (e.g., such that expandable structure 810 is sandwiched between support layer 812 and encapsulation layer 814. The method of FIG. 9 includes coupling electrodes 806 and electrode wires to support layer 812 (902). Electrodes 806 may be positioned at various axial and circumferential positions on flexible polymer packaging 808 corresponding to desired axial and circumferential positions around expandable structure 810. Electrode wires extend from electrodes 806 to elongated body 802 via strain relief 816 to deliver signals to and/or receive signals from lead 802. The method of FIG. 9 includes depositing encapsulation layer 814 on support layer 812 (904). Encapsulation layer 814 encapsulates the electrode wires, and in some examples, also embeds electrodes 806, such as by contacting lateral surfaces of electrodes 806.

In some examples of medical devices described herein, a medical device includes an electrode array configured to be positioned around an expandable structure, such as a stent, and coupled to an elongated member for positioning the electrode array. For example, an endovascular device may include an electrode array that may be formed using relatively simple and/or established manufacturing techniques, such as injection molding and laser cutting, to form a flexible polymer packaging and electrodes, respectively, prior to positioning the electrode array on the expandable structure.

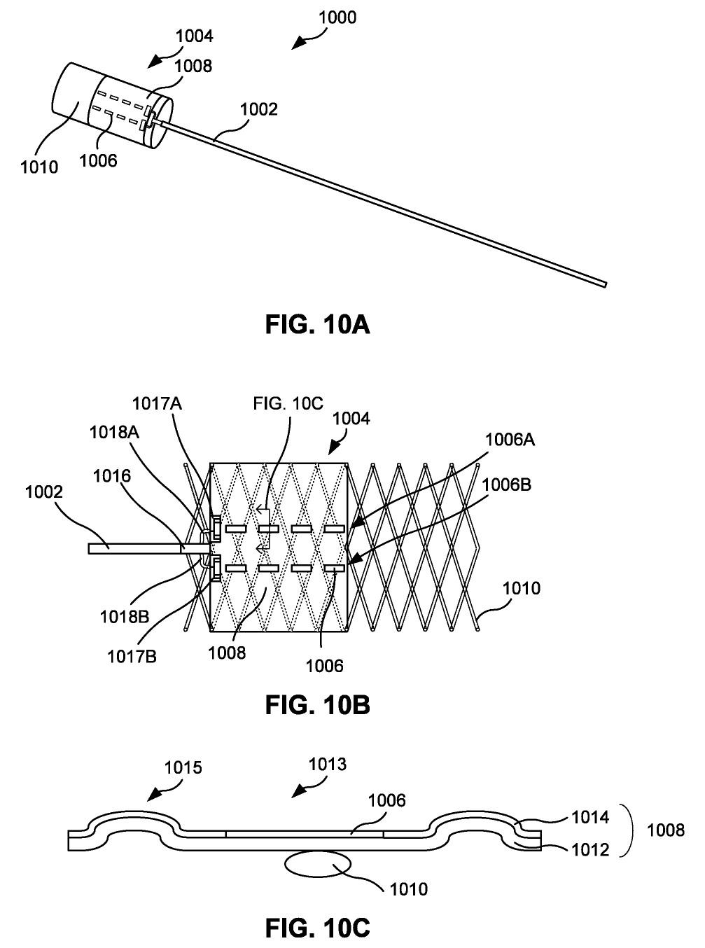

FIG. 10A is a perspective view of a distal portion of an example endovascular device 1000, while FIG. 10B is a top view of example endovascular device 1000 of FIG. 10A. Endovascular device 1000 includes elongated body 1002, electrode array 1004 including one or more electrodes 1006 and a flexible polymer packaging 1008, and an expandable structure 1010. Unless otherwise specified, elongated body 1002, electrodes 1006, flexible polymer packaging 1008, and expandable structure 1010 may be consistent with a general description of elongated body 23, electrodes 17, flexible polymer packaging 22, and expandable structure 19 of FIGS. 1A and 1B.

Elongated body 1002 is configured to be introduced into a blood vessel of a patient and remain at least partially in the blood vessel of the patient (e.g., a distal portion of elongated body 1002). Elongated body 1002 also includes array wires 1018A, 1018B configured to electrically couple to a respective contact pad 1017A, 1017B electrically coupled to electrodes 1006 and mechanically couple to flexible polymer packaging 1008.

In the example of FIGS. 10A and 10B, endovascular device 1000 includes a tubular expandable structure 1010. Tubular expandable structure 1010 may be similar to tubular expandable structure 510 of FIGS. 5A-C. In some examples, elongated body 1002 is mechanically coupled to expandable structure 1010 via connector 1016. Connector 1016 may be a ring or other structure configured to be crimped or welded to one or more structures of expandable structure 1010. In examples in which expandable structure 1010 is a stent, connector 1016 may be mechanically coupled to one or more struts of expandable structure 1010.

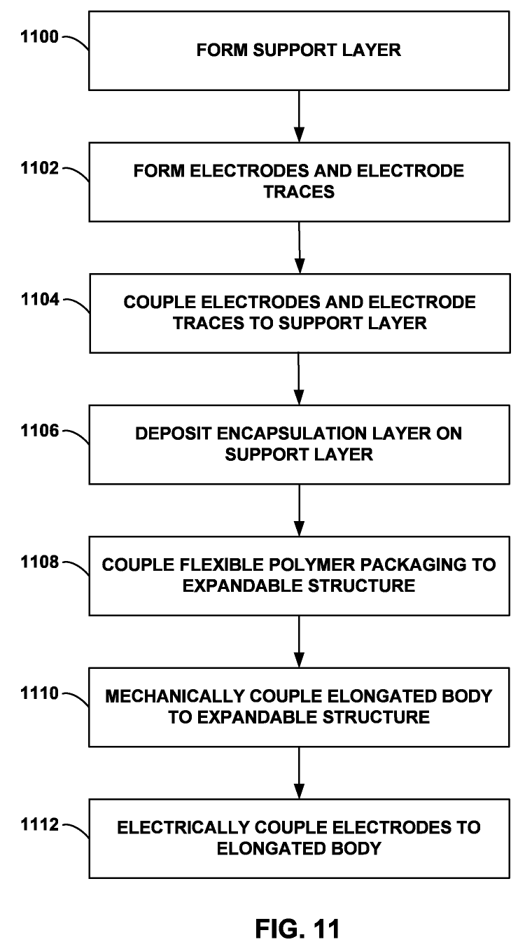

Endovascular device 1000 includes an electrode array 1004 coupled to expandable structure 1010. Electrode array 1004 includes flexible polymer packaging 1008 overlying at least a portion of expandable structure 1010. In some examples, flexible polymer packaging 1008 may be coupled to at least a portion of expandable structure 1010, such that flexible polymer packaging 1008 is secured to expandable structure 1010. For example, as will be described further in FIG. 11 below, electrode array 1004 may be formed separately from expandable structure 1010 and subsequently secured to expandable structure 1010. As a result, flexible polymer packaging 1008 may be formed using conventional fabrication techniques, such as injection molding, that enable particular structures in flexible polymer packaging 1008, such as will be described in FIG. 10C below.

Electrode array 1004 includes one or more electrodes 1006 electrically coupled to elongated body 1002, such that electrodes 1006 may receive therapy delivery signals to elongated body 1002 and/or send sensing signals to elongated body 1002. Electrodes 1006 are also coupled to flexible polymer packaging 1008. For example, electrodes 1006 may be adhered to and/or embedded in an outer surface of flexible polymer packaging 1008, either directly or through an adhesive. During operation, electrodes 1006 may contact an inner surface of the blood vessel to deliver electrical stimulation to tissue of the patient or sense a patient parameter from a location within the blood vessel.

Expandable structure 1010 is configured to expand and contract between a delivery configuration and a deployed configuration. To maintain a relatively constant circumferential position of electrodes 1006 around expandable structure 1010, flexible polymer packaging 1008 may be configured to expand and contract with expandable structure. Such expansion and contraction may result from compositional properties, such as flexibility or elasticity of the polymer, of flexible polymer packaging and/or structural features, such as accommodating structures, of electrode array 1004.

In some examples, flexible polymer packaging 1008 includes two or more electrode sections, such as may each be positioned at a particular circumferential position around expandable structure 1010. In the example of FIG. 10A, flexible polymer packaging 1008 includes a first electrode section coupled to a first portion of electrodes 1004A and a second electrode section coupled to a second portion of electrodes 1004B; however, in other example, electrode array 1004 may include additional electrode arrays and/or electrode arrays at other circumferential and/or longitudinal positions, such as around an entirety of expandable structure 1010. First portion of electrodes 1004A are electrically coupled to elongated body 502 via a first contact pad 1017A and a first array wire 1018A. Second portion of electrodes 1004B are electrically coupled to elongated body 1002 via a second contact pad 1017B and a second array wire 1018B.

To maintain the circumferential position of each portion of electrodes 1008A, 1008B, and/or to reduce bunching with such expansion or contraction, each electrode section may be configured to separate when expandable structure 1010 expands radially outward and move together when expandable structure 1010 contracts radially inward. To permit additional relative movement of the portions of electrodes 1006 beyond that provided by elastic properties of flexible polymer packaging 1008, flexible polymer packaging 1008 may include expandable sections between adjacent electrode sections, such as the first and second electrode sections. Each expandable sections is configured to permit the adjacent electrode sections to move relative to each other in response to axial or radial expansion or contraction of expandable structure 1010. For example, as will be illustrated in FIG. 10C below, each expandable section may be shaped to deform in response to expansion or contraction of expandable structure, such that a flexibility of the expandable section may be greater than a flexibility than the electrode sections. Array wires 1018A, 1018B may be sufficiently long to accommodate movement through a desired range of radial expansion and contraction of expandable structure 1010. In some examples, different portions of electrodes 1006 may have different properties. For example, the first portion of electrodes 1006A and the second portion of electrodes 1006B may have different polarities. In such examples, first and second portions of electrodes 1006A, 1006B may be separated by a particular spacing, such as from about three to about 7 millimeters.

FIG. 10C is a cross-sectional axis view diagram of the example endovascular device of FIG. 10A. Flexible polymer packaging 1008 may include different functional layers, including a support layer 1012 and an encapsulation layer 1014. Each of support layer 1012 and encapsulation layer 1014 may include a biocompatible polymer, such as biocompatible polymers described for support layer 212 and encapsulation layer 214 of FIG. 2D above.

Support layer 1012 is coupled to a portion of expandable structure 1010 and configured to secure flexible polymer packaging 1008, and therefore electrodes 1006, to expandable structure 1010. For example, support layer 1012 may attach to, conform to, and/or partially or fully embed portions of expandable structure 1010, such that support layer 1012 may adhere to expandable structure 1010. In the example of FIG. 10C, struts of expandable structure 1010 are underlying support layer 1012; however, in other examples, struts of expandable structure 1010 may be attached to support layer 1012, such as with another structure. In some examples, support layer 1012 may be relatively flexible, such that radial and/or axial expansion and contraction of expandable structure 1010 may cause expansion or contraction of support layer 1012 without support layer 1012 becoming detached from expandable structure 1010.

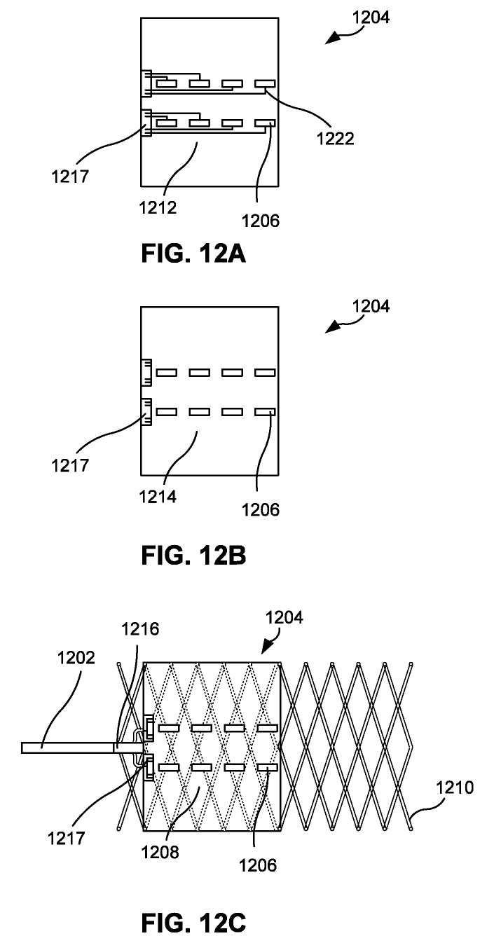

Encapsulation layer 1014 overlies support layer 1012. Encapsulation layer 1014 may be configured to encapsulate and/or secure various components of electrode array 1004, including electrodes 1006, electrode wires (not shown), or other components that may require insulation and/or be more securely attached by an additional layer. As will be described in FIGS. 11 and 12A-12C, after placement of electrodes 1006 on support layer 1012, electrodes 1006 may only be adhered by a single surface and/or components such as wires may be uninsulated. Encapsulation layer 1014 may be configured to encapsulate the electrode wires between electrodes 1006 and array wires 1018. In some examples, electrodes 1006 may be embedded in encapsulation layer 1014. For example, in FIG. 10C, encapsulation layer 1014 contacts the sides of electrode 1006, such that a surface of electrode 1006 is generally planar with a surface of encapsulation layer 1014. In other examples, encapsulation layer 1014 may only contact a portion of the sides of electrode 1006, or may overlap the side of electrode 1006 to partially cover edges of electrode 1006. Such contact may assist in securing electrodes 1006 to flexible polymer packaging 1008.

As discussed in FIGS. 10A and 10B above, flexible polymer packaging 1008 may include two or more electrode sections 1013 separated by an expandable section 1015. In the example of FIG. 10C, expandable section 1015 is C-shaped to accommodate expansion and contraction. For example, expandable section 1015 may elongated in response to expansion of expandable structure 1010 and compress in response to contraction of expandable structure 1010. In some examples, an amount of expansion permitted by expandable section 1015 may correspond to a desired diameter of expandable structure 1010 once expanded. For example, expandable section 1015 may elongate such that, once at the desired diameter, an outer surface of electrode array 1004 may be substantially smooth.

FIG. 11 is a flowchart of an example method for fabricating an endovascular device. The method of FIG. 11 will be described with respect to FIGS. 12A-12C, but may be used to form other endovascular devices. Unless otherwise noted, components of FIGS. 12A-12C may be operably and/or structurally similar to similarly numbered components of FIGS. 10A-10C.

The method of FIG. 11 includes forming a flexible polymer packaging 1208. In examples in which flexible polymer packaging 1208 includes two or more layers, the method of FIG. 11 includes forming a support layer 1212 (1100). Flexible polymer packaging 1208 may be formed separately from expandable structure 1210, such that flexible polymer packaging 1208 may be structurally rigid or semi-rigid to provide support for receiving electrodes 1206 independent of expandable structure 1210. Support layer 1212 may provide flexible polymer packaging 1208 with such support. Support layer 1212 may be a tubular sleeve configured to fit around expandable structure 1210.

As described in FIGS. 10A-10C, flexible polymer packaging 1208 may include two or more electrode sections separated by an expandable section. In such examples, support layer 1212 may define a general form of the expandable section. For example, support layer 1212 may include a C-shaped structure, or alternative structure, configured to permit relative movement between adjacent electrode sections in response to expansion or contraction of expandable structure 1210. In some examples, support layer 1212 is formed via injection molding.

The method of FIG. 11 includes forming electrodes 1206 and electrode traces 1222 (1102). In some examples, electrodes 1206 and electrode traces 1212 may be formed from a sheet of thin metal foil, such that electrodes 1206 and electrode traces 1212 each include metal foil. For example, a sheet of metal foil may be laser cut to form electrodes 1206 that are monolithic (or unitary) with a corresponding electrode trace 1212. As will be described below, such monolithic structures formed of metal foil may be thin and capable of being precisely positioned on support layer 1212.

The method of FIG. 11 includes coupling electrodes 1206 and electrode traces 1222 to support layer 1212 (1104). FIG. 12A is a top view of a plurality of electrodes 1206 and electrode traces 1222 overlying support layer 1212. To couple electrodes 1206 and electrode traces 1222 to support layer 1212, electrodes 1206 and electrode traces 1222 may be positioned and adhered at various positions on support layer 1212 that result in positioning at various axial and circumferential positions around expandable structure 1210. Electrode traces 1222 extend from electrodes 1206 to contact pad 1217 to electrically couple electrodes 1206 to contact pad 1217 and deliver signals to and/or receive signals from elongated body 1202 via contact pad 1217. In some examples, positioning electrodes 1206 may include positioning different portions of electrodes 1206 on different electrode sections of support layer 1212. The portions of electrodes 1206 may be separated by a particular spacing, such as from about 3 millimeters to about 7 millimeters once positioned around expandable structure 1210.

The method of FIG. 11 includes depositing encapsulation layer 1214 on support layer 1212 after coupling electrodes 1206 and electrode traces 1222 (1106), such that electrodes 1206 embedded in the encapsulation layer and most of electrode traces 1222 are covered by encapsulation layer 1214. FIG. 12B is a top view of an encapsulation layer overlying the support layer of FIG. 12A. In some examples, encapsulation layer 1214 may be deposited over electrodes 1206, and various openings may be formed in encapsulation layer 1214, such as through cutting or localized melting. In other examples, openings may be included in encapsulation layer 1214, thereby exposing surfaces of electrodes 1206. In some examples, encapsulation layer 1214 is a fluid that is extruded onto support layer 1212, thereby covering electrode wires 1222 and flow around electrodes 1206. Encapsulation layer 1214 encapsulates electrode traces 1222, and in some examples, may also embed electrodes 1206, such as by contacting lateral surfaces and/or top surfaces of electrodes 1206. The resulting electrode array 1204 may be coupled to additional structures, such as expandable structure 1210 and elongated body 1202.

The method of FIG. 11 includes coupling flexible polymer packaging 1208 to at least a portion of tubular expandable structure 1210 (1108). FIG. 12C is a top view of a flexible polymer packaging coupled to an expandable member. In some examples, expandable structure 1210 is a tubular stent that includes a plurality of interconnected struts, such that at least a portion of the plurality of interconnected struts are coupled to flexible polymer packaging 1208.

The method of FIG. 11 includes mechanically coupling elongated body 1202 to expandable structure 1210 (1110). For example, lead connector 1216 may be welded to one or more structures of expandable structure 1210. In some examples, elongated body 1202 may also be mechanically coupled to flexible polymer packaging 1208, such as via array wires 1218

The method of FIG. 11 includes electrically coupling electrodes 1206 to elongated body 1202 (1112). For example, array wires 1218 coupled to elongated body 1202 may be electrically coupled to electrical traces 1222 on contact pad 1217.

The examples described herein may be combined in any permutation or combination.

Example 1A: A medical device includes an elongated body configured to be introduced into a patient; a non-tubular expandable structure configured to bend around a longitudinal axis of the expandable structure; and an electrode array includes a flexible polymer packaging overlying at least a portion of the expandable structure; and one or more electrodes electrically coupled to the elongated body and coupled to the flexible polymer packaging.

Example 2A: The medical device of example 1A, wherein the expandable structure is configured to self-deploy from a delivery configuration to a deployed configuration to position the one or more electrodes to deliver electrical stimulation to tissue of the patient or sense a patient parameter from a location within a blood vessel.

Example 3A: The medical device of example 2A, wherein the expandable structure comprises a shape memory metal.

Example 4A: The medical device of any of examples 1A through 3A, wherein the expandable structure is a flexible metal template.

Example 5A: The medical device of example 4A, wherein the flexible metal template comprises a plurality of projections extending from a central spine, and wherein the elongated body is coupled to the flexible metal template at the central spine.

Example 6A: The medical device of any of examples 1A through 5A, wherein the elongated body is mechanically coupled to the expandable structure.

Example 7A: The medical device of any of examples 1A through 6A, wherein the flexible polymer packaging comprises: a support layer coupled to the portion of the expandable structure; and an encapsulation layer overlying the support layer and coupled to the one or more electrodes.

Example 8A: The medical device of example 7A, wherein the one or more electrodes are embedded in the encapsulation layer.

Example 9A: The medical device of any of examples 7A and 8A, wherein the support layer comprises at least one of polyurethane or silicone.

Example 10A: The medical device of any of examples 7A through 9A, wherein the encapsulation layer comprises at least one of polyurethane or silicone.

Example 11A: The medical device of any of examples 1A through 10A, wherein the non-tubular expandable structure is configured to bend such that the electrode array faces radially outward.

Example 12A: The medical device of any of examples 1A through 11A, wherein the non-tubular expandable structure is configured to bend such that the electrode array faces radially inward.

Example 13A: The medical device of any of examples 1A through 12A, wherein the expandable structure is a planar stent preform configured to bend from a planar form into a cylindrical form.

Example 14A: The medical device of example 13A, wherein the medical device is an endovascular device configured to self-deploy from a delivery configuration to a deployed configuration to position the one or more electrodes to deliver electrical stimulation to tissue of the patient or sense a patient parameter from a location within a blood vessel.

Example 15A: The medical device of any of examples 1A through 14A, wherein the medical device is a subcutaneous device configured to self-deploy from a delivery configuration to a deployed configuration to position the one or more electrodes to deliver electrical stimulation to tissue of the patient or sense a patient parameter from a location beneath skin of the patient.

Example 16A: A medical device therapy system includes the medical device of any of examples 1A to 15A; and an implantable neurostimulator electrically coupled to the medical device.

Example 17A: A method for forming a medical device includes forming a flexible polymer packaging on at least a portion of an expandable structure, wherein the expandable structure is configured to bend around an axis; and coupling one or more electrodes to the flexible polymer packaging, wherein the one or more electrodes are electrically coupled to an elongated body.

Example 18A: The method of example 17A, wherein the expandable structure is a planar stent preform configured to bend from a planar form into a cylindrical form.

Example 19A: The method of any of examples 17A and 18A, wherein the expandable structure is configured to self-deploy from a delivery configuration to a deployed configuration to position the one or more electrodes to deliver electrical stimulation to tissue of the patient or sense a patient parameter from a location within the blood vessel.

Example 20A: The method of example 19A, wherein the expandable structure comprises a shape memory metal.

Example 21A: The method of any of examples 17A through 20A, further comprising forming the expandable structure from a flexible metal sheet.

Example 22A: The method of example 21A, further comprising machining the flexible metal sheet to form a central spine and a plurality of projections extending from the central spine.

Example 23A: The method of any of examples 17A through 22A, further comprising mechanically coupling the elongated body to the expandable structure.

Example 24A: The method of any of examples 17A through 23A, wherein forming the flexible polymer packaging comprises: prior to positioning the one or more electrodes, depositing a support layer on the portion of the expandable structure; and after positioning the one or more electrodes, depositing an encapsulation layer on the support layer.

Example 25A: The method of example 24A, wherein the encapsulation layer embeds the one or more electrodes.

Example 26A: The method of any of examples 24A and 25A, wherein the support layer comprises at least one of polyurethane or silicone.

Example 27A: The method of any of examples 24A through 26A, wherein the encapsulation layer comprises at least one of polyurethane or silicone.

Example 28A: The method of any of examples 17A through 27A, wherein the non-tubular expandable structure is configured to bend such that the electrode array faces radially outward.

Example 29A: The method of any of examples 17A through 28A, wherein the non-tubular expandable structure is configured to bend such that the electrode array faces radially inward.

Example 1B: An endovascular device includes an elongated body configured to be introduced into a blood vessel of a patient; a tubular expandable structure; and an electrode array includes a flexible polymer packaging molded to at least a portion of the expandable structure; and one or more electrodes electrically coupled to the elongated body and coupled to the flexible polymer packaging.

Example 2B: The endovascular device of example 1B, wherein the expandable structure is a tubular stent comprising a plurality of interconnected struts, and wherein at least a portion of the plurality of interconnected struts are embedded in the flexible polymer packaging.

Example 3B: The endovascular device of any of examples 1B and 2B, wherein the expandable structure is configured to expand from a delivery configuration to a deployed configuration to position the one or more electrodes to deliver electrical stimulation to tissue of the patient or sense a patient parameter from a location within the blood vessel.

Example 4B: The endovascular device of any of examples 1B through 3B, wherein the elongated body is mechanically coupled to the expandable structure.

Example 5B: The endovascular device of example 4B, wherein the elongated body is welded to the expandable structure.

Example 6B: The endovascular device of any of examples 1B through 5B, wherein the flexible polymer packaging comprises: a support layer coupled to the portion of the expandable structure; and an encapsulation layer overlying the support layer, wherein the one or more electrodes are embedded in the encapsulation layer.

Example 7B: The endovascular device of example 6B, wherein the support layer comprises at least one of polyurethane or silicone.

Example 8B: The endovascular device of any of examples 6B and 7B, wherein the encapsulation layer comprises at least one of polyurethane or silicone.

Example 9B: The endovascular device of any of examples 1B through 8B, wherein the electrode array comprises: a first electrode array includes a first portion of the flexible polymer packaging; and a first portion of the one or more electrodes coupled to the first portion of the flexible polymer packaging and electrically coupled to the elongated body via a first wire; and a second electrode array includes a second portion of the flexible polymer packaging; and a second portion of the one or more electrodes coupled to the second portion of the flexible polymer packaging and electrically coupled to the elongated body via a second wire.

Example 10B: The endovascular device of example 9B, wherein the first electrode array and the second electrode array are configured to move relative to each other in response to expansion or contraction of the expandable structure.

Example 11B: The endovascular device of any of examples 9B and 10B, wherein the first portion of the one or more electrodes and the second portion of the one or more electrodes have a different polarity, and wherein the first electrode array and the second electrode array are separated by a spacing less than about one millimeter.

Example 12B: A medical device therapy system includes the endovascular device of any of examples 1B to 11B; and an implantable neurostimulator electrically coupled to the endovascular device.

Example 13B: A method for forming a medical device includes molding a flexible polymer packaging to at least a portion of a tubular expandable structure; and coupling one or more electrodes to the flexible polymer packaging, wherein the one or more electrodes are electrically coupled to an elongated body.

Example 14B: The method of example 13B, wherein the expandable structure is a tubular stent comprising a plurality of interconnected struts, and wherein at least a portion of the plurality of interconnected struts are embedded in the flexible polymer packaging.

Example 15B: The method of any of examples 13B and 14B, wherein the expandable structure is configured to expand from a delivery configuration to a deployed configuration to position the one or more electrodes to deliver electrical stimulation to tissue of the patient or sense a patient parameter from a location within the blood vessel.

Example 16B: The method of any of examples 13B through 15B, further comprising mechanically coupling the elongated body to the expandable structure.

Example 17B: The method of example 16B, wherein mechanically coupling the elongated body to the expandable structure comprises welding the elongated body to the expandable structure.

Example 18B: The method of any of examples 13B through 17B, wherein forming the flexible polymer packaging comprises: prior to coupling the one or more electrodes, depositing a support layer on the portion of the expandable structure; and after coupling the one or more electrodes, depositing an encapsulation layer on the support layer, wherein the one or more electrodes are embedded in the encapsulation layer.

Example 19B: The method of any of examples 13B through 18B, wherein the support layer comprises at least one of polyurethane or silicone.

Example 20B: The method of any of examples 13B through 19B, wherein the encapsulation layer comprises at least one of polyurethane or silicone.

Example 21B: The method of any of examples 13B through 20B, wherein the electrode array comprises: a first electrode array includes a first portion of the flexible polymer packaging; and a first portion of the one or more electrodes coupled to the first portion of the flexible polymer packaging and electrically coupled to the elongated body via a first wire; and a second electrode array includes a second portion of the flexible polymer packaging; and a second portion of the one or more electrodes coupled to the second portion of the flexible polymer packaging and electrically coupled to the elongated body via a second wire.

Example 22B: The method of example 21B, wherein the first electrode array and the second electrode array are configured to move relative to each other in response to expansion or contraction of the expandable structure.

Example 23B: The method of any of examples 21B and 22B, wherein the first portion of the one or more electrodes and the second portion of the one or more electrodes have a different polarity, and wherein the first electrode array and the second electrode array are separated by a spacing less than about one millimeter.

Example 1C: An endovascular device includes an elongated body configured to be introduced into a blood vessel of a patient; an expandable structure mechanically coupled to the elongated body and configured to be positioned within a stent; and an electrode array includes a flexible polymer packaging coupled to at least a portion of the expandable structure; and one or more electrodes electrically coupled to the elongated body and coupled to the flexible polymer packaging.

Example 2C: The endovascular device of example 1C, wherein the expandable structure is a helical expandable structure.

Example 3C: The endovascular device of any of examples 1C and 2C, wherein the expandable structure is configured to expand from a delivery configuration to a deployed configuration to position the one or more electrodes to deliver electrical stimulation to tissue of the patient or sense a patient parameter from a location within the blood vessel.

Example 4C: The endovascular device of any of examples 1C through 3C, wherein the flexible polymer packaging comprises: a support layer coupled to the portion of the expandable structure; and an encapsulation layer overlying the support layer, wherein the one or more electrodes are embedded in the encapsulation layer.

Example 5C: The endovascular device of example 4C, wherein the support layer comprises at least one of polyurethane or silicone.

Example 6C: The endovascular device of any of examples 4C and 5C, wherein the encapsulation layer comprises at least one of polyurethane or silicone.

Example 7C: The endovascular device of any of examples 1C through 6C, wherein the expandable structure comprises a strain relief configured to expand along a longitudinal axis in response to expansion or contraction of the stent.

Example 8C: The endovascular device of any of examples 1C through 7C, wherein the flexible polymer packaging is configured to conform to a surface of the stent.

Example 9C: The endovascular device of any of examples 1C through 8C, wherein the stent defines a longitudinal axis, and wherein the flexible polymer packaging is configured to expand along and around the longitudinal axis.

Example 10C: The endovascular device of example 9C, wherein the flexible polymer packaging is configured to expand at least ten percent along and around the longitudinal axis.

Example 11C: The endovascular device of any of examples 1C through 10C, wherein the electrode array comprises: a first electrode array includes a first portion of the flexible polymer packaging; and a first portion of the one or more electrodes coupled to the first portion of the flexible polymer packaging; and a second electrode array includes a second portion of the flexible polymer packaging; and a second portion of the one or more electrodes coupled to the second portion of the flexible polymer packaging.

Example 12C: The endovascular device of example 11C, wherein the first electrode array and the second electrode array are configured to move relative to each other in response to expansion or contraction of the expandable structure.

Example 13C: A medical device therapy system includes the endovascular device of any of examples 1C to 12C; and an implantable neurostimulator electrically coupled to the endovascular device.

Example 14C: A method for forming a medical device includes forming a flexible polymer packaging on at least a portion of an expandable structure wherein the expandable structure is mechanically coupled to an elongated body and configured to be positioned within a stent; and coupling one or more electrodes to the flexible polymer packaging, wherein the one or more electrodes are electrically coupled to the elongated body.

Example 15C: The method of example 14C, wherein the expandable structure is a helical expandable structure.

Example 16C: The method of any of examples 14C and 15C, wherein the expandable structure is configured to expand from a delivery configuration to a deployed configuration to position the one or more electrodes to deliver electrical stimulation to tissue of the patient or sense a patient parameter from a location within the blood vessel.

Example 17C: The method of any of examples 14C through 16C, wherein forming the flexible polymer packaging comprises: prior to coupling the one or more electrodes, depositing a support layer on the portion of the expandable structure; and after coupling the one or more electrodes, depositing an encapsulation layer on the support layer, wherein the one or more electrodes are embedded in the encapsulation layer.

Example 18C: The method of example 17C, wherein the support layer comprises at least one of polyurethane or silicone.

Example 19C: The method of any of examples 17C and 18C, wherein the encapsulation layer comprises at least one of polyurethane or silicone.

Example 20C: The method of any of examples 14C through 19C, wherein the expandable structure comprises a strain relief configured to expand along a longitudinal axis in response to expansion or contraction of the stent.

Example 21C: The method of any of examples 14C through 20C, wherein the flexible polymer packaging is configured to conform to a surface of the stent.

Example 22C: The method of any of examples 14C through 21C, wherein the stent defines a longitudinal axis, and wherein the flexible polymer packaging is configured to expand along and around the longitudinal axis.

Example 23C: The method of example 22C, wherein the flexible polymer packaging is configured to expand at least ten percent along and around the longitudinal axis.

Example 24C: The method of any of examples 14C through 23C, wherein the electrode array comprises: a first electrode array includes a first portion of the flexible polymer packaging; and a first portion of the one or more electrodes coupled to the first portion of the flexible polymer packaging; and a second electrode array includes a second portion of the flexible polymer packaging; and a second portion of the one or more electrodes coupled to the second portion of the flexible polymer packaging.

Example 25C: The method of example 24C, wherein the first electrode array and the second electrode array are configured to move relative to each other in response to expansion or contraction of the expandable structure.

Example 1D: An endovascular device includes an elongated body configured to be introduced into a blood vessel of a patient; a tubular expandable structure; and an electrode array includes a flexible polymer packaging coupled to at least a portion of the expandable structure; and one or more electrodes electrically coupled to the elongated body and coupled to the flexible polymer packaging.

Example 2D: The endovascular device of example 1D, wherein the tubular expandable structure is a tubular stent comprising a plurality of interconnected struts, and wherein at least a portion of the plurality of interconnected struts are coupled to the flexible polymer packaging.

Example 3D: The endovascular device of any of examples 1D and 2D, wherein the tubular expandable structure is configured to expand from a delivery configuration to a deployed configuration to position the one or more electrodes to deliver electrical stimulation to tissue of the patient or sense a patient parameter from a location within the blood vessel.

Example 4D: The endovascular device of any of examples 1D through 3D, wherein the elongated body is mechanically coupled to the tubular expandable structure.

Example 5D: The endovascular device of example 4D, wherein the elongated body is welded to the tubular expandable structure.

Example 6D: The endovascular device of any of examples 1D through 5D, wherein the flexible polymer packaging comprises: a support layer coupled to the portion of the tubular expandable structure; and an encapsulation layer overlying the support layer, wherein the one or more electrodes are embedded in the encapsulation layer.

Example 7D: The endovascular device of example 6D, wherein the support layer comprises at least one of polyurethane or silicone.

Example 8D: The endovascular device of any of examples 6D and 7D, wherein the encapsulation layer comprises at least one of polyurethane or silicone.

Example 9D: The endovascular device of any of examples 1D through 8D, further comprising one or more electrode traces, wherein each electrode trace is monolithic with a corresponding electrode.

Example 10D: The endovascular device of example 9D, wherein each of the one or more electrodes and the one or more electrode traces comprise a metal foil.

Example 11D: The endovascular device of any of examples 1D through 10D, wherein the flexible polymer packaging comprises: a first electrode section coupled to a first portion of the one or more electrodes; a second electrode section coupled to a second portion of the one or more electrodes; and an expandable section separating the first and second electrode sections and configured to permit the first and second electrode sections to move relative to each other in response to expansion or contraction of the tubular expandable structure.

Example 12D: The endovascular device of example 11D, wherein a flexibility of the expandable section is greater than a flexibility than the first and second electrode sections.

Example 13D: A medical device therapy system includes the endovascular device of any of examples 1D to 12D; and an implantable neurostimulator electrically coupled to the endovascular device.

Example 14D: A method for forming a medical device includes coupling one or more electrodes to a flexible polymer packaging to form an electrode array, wherein the one or more electrodes are electrically coupled to an elongated body; coupling the flexible polymer packaging to at least a portion of a tubular expandable structure; and electrically coupling the one or more electrodes to an elongated body configured to be introduced into a blood vessel of a patient.

Example 15D: The method of example 14D, wherein the tubular expandable structure is a tubular stent comprising a plurality of interconnected struts, and wherein at least a portion of the plurality of interconnected struts are coupled to the flexible polymer packaging.

Example 16D: The method of any of examples 14D and 15D, wherein the tubular expandable structure is configured to expand from a delivery configuration to a deployed configuration to position the one or more electrodes to deliver electrical stimulation to tissue of the patient or sense a patient parameter from a location within the blood vessel.

Example 17D: The method of any of examples 14D through 16D, further comprising mechanically coupling the elongated body to the expandable structure.

Example 18D: The method of any of examples 14D through 17D, further comprising forming the flexible polymer packaging.

Example 19D: The method of example 18D, wherein forming the flexible polymer packaging comprises: prior to coupling the one or more electrodes, forming a support layer; and after coupling the one or more electrodes, depositing an encapsulation layer on the support layer, wherein the one or more electrodes are embedded in the encapsulation layer.

Example 20D: The method of example 19D, wherein the support layer comprises at least one of polyurethane or silicone.

Example 21D: The method of any of examples 19D and 20D, wherein the support layer is formed via injection molding.

Example 22D: The method of any of examples 19D through 21D, wherein the support layer comprises a tubular sleeve configured to fit around the expandable structure.

Example 23D: The method of any of examples 19D through 22D, wherein the encapsulation layer comprises at least one of polyurethane or silicone.

Example 24D: The method of any of examples 14D through 23D, wherein coupling the one or more electrodes to the flexible polymer packaging comprises coupling the one or more electrodes and one or more electrode traces to the flexible polymer packaging, and wherein each electrode trace is monolithic with a corresponding electrode.

Example 25D: The method of example 24D, wherein each of the one or more electrodes and the one or more electrode traces comprise a metal foil.

Example 26D: The method of example 25D, further comprising forming the one or more electrodes and the one or more electrode traces from a sheet of metal foil.

Example 27D: The method of any of examples 14D through 26D, wherein the flexible polymer packaging comprises: a first electrode section coupled to a first portion of the one or more electrodes; a second electrode section coupled to a second portion of the one or more electrodes; and an expandable section separating the first and second electrode sections and configured to permit the first and second electrode sections to move relative to each other in response to expansion or contraction of the expandable structure.

Example 28D: The method of example 27D, wherein a flexibility of the expandable section is greater than a flexibility than the first and second electrode sections.

Various aspects of the disclosure have been described. These and other aspects are within the scope of the following claims.

What is claimed is:

1. A medical device comprising:
an elongated body configured to be introduced into a patient;
a non-tubular expandable structure configured to bend around a longitudinal axis of the expandable structure; and
an electrode array comprising:
a flexible polymer packaging overlying at least a portion of the expandable structure; and
one or more electrodes electrically coupled to the elongated body and coupled to the flexible polymer packaging.

2. The medical device of claim 1, wherein the expandable structure is configured to self-deploy from a delivery configuration to a deployed configuration to position the one or more electrodes to deliver electrical stimulation to tissue of the patient or sense a patient parameter from a location within a blood vessel.

3. The medical device of claim 2, wherein the expandable structure comprises a shape memory metal.

4. The medical device of claim 1, wherein the expandable structure is a flexible metal template.

5. The medical device of claim 4,
wherein the flexible metal template comprises a plurality of projections extending from a central spine, and
wherein the elongated body is coupled to the flexible metal template at the central spine.

6. The medical device of claim 1, wherein the elongated body is mechanically coupled to the expandable structure.

7. The medical device of claim 1, wherein the flexible polymer packaging comprises:
a support layer coupled to the portion of the expandable structure; and
an encapsulation layer overlying the support layer and coupled to the one or more electrodes.

8. The medical device of claim 7, wherein the one or more electrodes are embedded in the encapsulation layer.

9. The medical device of claim 1, wherein the non-tubular expandable structure is configured to bend such that the electrode array faces radially outward.

10. The medical device of claim 1, wherein the non-tubular expandable structure is configured to bend such that the electrode array faces radially inward.

11. The medical device of claim 1, wherein the expandable structure is a planar stent preform configured to bend from a planar form into a cylindrical form.

12. The medical device of claim 11, wherein the medical device is an endovascular device configured to self-deploy from a delivery configuration to a deployed configuration to position the one or more electrodes to deliver electrical stimulation to tissue of the patient or sense a patient parameter from a location within a blood vessel.

13. The medical device of claim 1, wherein the medical device is a subcutaneous device configured to self-deploy from a delivery configuration to a deployed configuration to position the one or more electrodes to deliver electrical stimulation to tissue of the patient or sense a patient parameter from a location beneath skin of the patient.

14. A medical device therapy system comprising:
a medical device comprising:
an elongated body configured to be introduced into a patient;
a non-tubular expandable structure configured to bend around a longitudinal axis of the expandable structure; and
an electrode array comprising:
a flexible polymer packaging overlying at least a portion of the expandable structure; and
one or more electrodes electrically coupled to the elongated body and coupled to the flexible polymer packaging; and
an implantable neurostimulator electrically coupled to the medical device.

15. A method for forming a medical device, comprising:
forming a flexible polymer packaging on at least a portion of an expandable structure, wherein the expandable structure is configured to bend around an axis; and
coupling one or more electrodes to the flexible polymer packaging, wherein the one or more electrodes are electrically coupled to an elongated body.

16. The method of claim 15, wherein the expandable structure is a planar stent preform configured to bend from a planar form into a cylindrical form.

17. The method of claim 15, further comprising forming the expandable structure from a flexible metal sheet.

18. The method of claim 17, further comprising machining the flexible metal sheet to form a central spine and a plurality of projections extending from the central spine.

19. The method of claim 15, further comprising mechanically coupling the elongated body to the expandable structure.

20. The method of claim 15, wherein forming the flexible polymer packaging comprises:
prior to positioning the one or more electrodes, depositing a support layer on the portion of the expandable structure; and
after positioning the one or more electrodes, depositing an encapsulation layer on the support layer.
